(19) United States
(12) Patent Application Publication
(10) Pub. No.: US 2025/0268996 A1
(43) Pub. Date: Aug. 28, 2025

(54) METHODS OF AND COMPOSITIONS FOR TREATMENT OF UPPER LIMB SPASTICITY

(71) Applicant: REVANCE THERAPEUTICS, INC., Newark, CA (US)

(72) Inventors: Roman Rubio, Redwood City, CA (US); Daniel Snyder, Mountain View, CA (US)

(21) Appl. No.: 18/278,169

(22) PCT Filed: Feb. 21, 2022
(86) PCT No.: PCT/US2022/017170

§ 371 Date: Aug. 21, 2023

Related U.S. Application Data
(60) Provisional application No. 63/151,795, filed on Feb. 21, 2021.

(51) Int. Cl. A61K 38/48; A61K 9/00; A61P 25/14

(52) U.S. Cl. CPC A61K 38/4893; A61K 9/0019; A61P 25/14

(57) ABSTRACT

This invention provides compostions for and methods of treating upper limb spasticity (ULS) with novel injectable compositions comprising botulinum toxin that may be administered to a subject suffering therefrom. The injectable compositions and methods in which these compositions are used provide novel and advantageous treatments which result in long duration of effect, for example, a duration of effect for 16 weeks and longer, such as 20 weeks, 24 weeks, 28 weeks, 32 weeks, or 36 weeks.

### SEQUENCE LISTING

The instant application contains a Sequence Listing which has been submitted electronically in ASCII format and is hereby incorporated by reference in its entirety. Said ASCII copy, created on ______, is named ______.txt and is ______ bytes in size.

### FIELD OF THE INVENTION

This invention relates to methods of and compositions for treating upper limb spasticity (ULS) with an injectable composition comprising botulinum toxin that may be administered to a subject suffering from such malady. The injectable compositions and methods in which these compositions are used provide novel and advantageous treatments which result in a long duration of effect, for example, a duration of effect of 16 weeks.

### BACKGROUND OF THE INVENTION

Spasticity is an involuntary overactivity of muscles that occurs following upper motor neuron damage to the brain or spinal cord, usually by a stroke, disease, or injury. Spasticity of the upper limb (ULS) is a condition that affects muscles involved in the movement of the upper limbs, particularly movement of the shoulders, arms and hands. ULS is common after stroke and can cause deformity, pain and reduced function. For those with such condition, muscles in the upper arm are stiff and flexed. Sometimes, the upper limb muscles will twitch or move in an uncontrolled manner, called a spasm. Spasm may also be associated with muscle hypertonicity. The condition is not life-threatening, but it can be painful and can make life's daily tasks, such as getting dressed or bathing, difficult.

Usually, treatment of ULS is only required if the spasticity causes disruptive or painful symptoms or limits function. The clinical pattern of clenched first is especially likely to affect function and result in complications such as palmar skin breakdown, infection, and soft tissue contracture development. The management of ULS includes non-pharmacologic treatment, such as physiotherapy and splinting, and pharmacologic treatment, including oral medication (such as muscle relaxants) and treatment with botulinum toxin, particularly botulinum toxin type A (BONTA), administered through intramuscular injections. Multiple therapies are often used concomitantly. Treatment goals in the management of ULS include relief from pain and muscle spasms, functional improvement in both active and passive dimensions, avoiding progression of impairment, and improving aesthetic and postural appearance. There may be little opportunity to restore active function, but improving the ease of caring for the affected limb, for example, in washing and dressing, can nevertheless have a significant impact on caregiver burden, and can potentially have significant cost benefits in terms of reducing the time taken, or the number of people required, to perform care tasks. BoNTA injections are recommended as a safe and effective treatment for the reduction of muscle spasticity and improvement of passive function in patients with ULS. Botulinum toxin injections are currently the first-line pharmacological option for treatment of focal ULS.

Botulinum toxin can help block the communication between the nerve and the muscle and may alleviate abnormal movements and postures. The number of injections is typically based on the severity of the spasticity. Doctors injecting the toxin may select the muscles to be injected by observing abnormal bending at joints or movements and feeling for the muscle spasm or by using electromyography (EMG) to measure muscle activity. Each muscle affected by spasticity typically has to be injected separately. As such, based on the diffusion characteristics of currently available toxin formulations, there is a limit to the total quantity of toxin that can be injected into the body at one time. While the treatment for ULS involves regular neurological intervention, which takes effect over a period of 4-7 days or longer after injection, the response to the treatment with botulinum toxin typically wears off after a 12 week period, often as early as at 10 weeks, requiring the person suffering from ULS to be injected again. Therefore, a durable, longer acting treatment requiring fewer neurological interventions would be desirable.

### SUMMARY OF THE INVENTION

In one of its aspects, the invention relates to a method for producing a biologic effect in the treatment of ULS by injecting an effective amount, preferably a therapeutically effective amount, of the compositions of this invention to a subject or patient in need of such treatment. Such biologic effect can last for at least 16 weeks or longer extending the tme between subsequent treatments.

An embodiment of the disclosure comprises a method of treating ULS in an individual in need thereof by administering to the individual an injection of a composition comprising: a botulinum toxin component and a positively charged carrier; and a pharmaceutically acceptable diluent for injection; wherein the botulinum toxin is administered to the individual in an amount from about 250 U to about 1000 U; wherein the positively charged carrier is non-covalently associated with the botulinum toxin component; and wherein injection of the composition provides a treatment having at least about a four month to about a 9 months duration of effect in reducing the symptoms of ULS, thereby extending treatment interval duration for the individual. In embodiments, the positively charged carrier can be a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RKKRRQRRR-(gly)q (SEQ ID NO: 3), wherein the subscripts p and q are each independently an integer of from 0 to 20

In another of its aspects, this invention provides injectable compositions comprising botulinum toxin non-covalently associated with a positively charged carrier molecule used to treat ULS. In preferred embodiments, the compositions of the invention possess one or more advantages over conventional commercial botulinum toxin formulations, such as BOTOX® or MYOBLOC®. For instance, in certain embodiments, the compositions may exhibit one or more advantages over conventional injectable botulinum formulations, including reduced antigenicity, a reduced tendency to undergo diffusion into surrounding tissue following injection, increased duration of clinical efficacy or enhanced potency relative to conventional botulinum toxin formulations, faster onset of clinical efficacy, and/or improved stability.

While not wishing to be bound by theory, the positively charged carriers are non-native molecules (i.e., molecules not found in botulinum toxin complexes obtained from Clostridium botulinum bacteria) that can improve toxin diffusion through tissues in the treatment of ULS. The positively charged carriers can associate non-covalently with the toxin and are believed to act as penetration enhancers that improve the ability of the toxin to reach target structures after injection. Furthermore, the positively charged carriers may increase the stability of the toxin prior to and after injection. By way of example, the The positively charged carriers can be cationic peptides, which have no inherent botulinum-toxin-like activity and which also contain one or more protein transduction domains as described herein.

The compositions comprising botulinum toxin component can be the 150 kD neurotoxin itself, or the neurotoxin with some, but not all, of the native complex proteins).

Methods of the present disclosure provide effective doses and amounts of the compositions of this invention in the treatment of ULS that afford a long-lasting, sustained efficacy following administration by injection to a subject or patient in need of treatment. Such doses and amounts are preferably therapeutically effective doses and amounts that produce or result in a desired therapeutic effect in a subject to whom the doses and amounts are administered. In particular embodiments, a single treatment of a subject or patient with a composition of the invention comprising a botulinum toxin, such as botulinum toxin A, and a positively charged carrier, as described herein, in therapeutically effective dose amounts of about 250 U to about 500 U per subject, afford a response rate in the reduction of ULS symptoms for at least 16 weeks, at least 20 weeks, at least 24 weeks, at least 28 weeks, at least 32 weeks, at least 36 weeks.

The duration of effect provided by compositions described herein as well as by the described treatment methods and uses, affords significant advantages compared to the art. By way of example, subjects undergoing treatment with compositions containing botulinum toxin benefit from having a duration of effect following treatment to be of that reduces the need for follow up or repeat treatment, or extends the interval between treatments. Such a long, sustained duration of effect, which is achieved by even a single treatment with an effective dose and treatment regime of a product of the invention, for example, DAXI described in Example 1, permits fewer injections per treatment course for a subject. A prolonged duration effect from a single treatment with a product which has clear efficacy and safety, as provided by the inventive compositions and methods described herein, offer less discomfort, less cost as result of few treatments, and more convenience to subjects undergoing a course of treatment. Furthermore, a product that affords significant and sustained effects, which are maintained for at least a 16, 20, 24, 28, 32, or 36 week period following the single treatment of the product to a subject, provides a solution to an unmet need in the art for both practitioners and patients alike. Thus, the compositions and methods of the invention provide a solution to the problem of too frequent treatments and improve patients' overall well-being. Such prolonged duration of action provides for fewer treatments over an entire treatment course.

The disclosure described herein is directed to a method of administering botulinum toxin to achieve an extended duration therapeutic effect in an individual suffering from ULS, in which the method comprises administering by injection a dose of a sterile injectable composition into an area of the individual in need of treatment to achieve the therapeutic effect following a first treatment with the composition; wherein the composition comprises a botulinum toxin component and a positively charged carrier; wherein the botulinum toxin component is administered to the individual in a treatment dose of about 250 U to 1000 U; or more specifically, from about 250 U to 500 U or from about 500 U to 700 U or from about 600 U to 800 U or from about 800 U to 1000 U, wherein the positively charged carrier is non-covalently associated with the botulinum toxin, botulinum toxin complex, or reduced botulinum toxin complex component; and a pharmaceutically acceptable diluent suitable for injection; and wherein the first treatment dose of the composition administered by injection to the individual achieves the extended duration therapeutic effect of at least 16 weeks, 20 weeks, 24 weeks, 28 weeks, 32 weeks, or 36 weeks before a second or subsequent treatment dose is administered. In embodiments, the positively charged carrier can comprise a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RKKRRQRRR-(gly)q (SEQ ID NO: 3), wherein the subscripts p and q are each independently an integer of from 0 to 20

The disclosure described herein is directed to a sterile injectable composition for treating one or more types (e.g., clinical presentation patterns) of ULS comprising a botulinum toxin, a botulinum toxin complex, or a reduced botulinum toxin complex in a dosage amount selected from more than 250 U, from 250 U to 500 U, 500-750 U, or 750-1000 U; and a positively charged carrier comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RKKRRQRRR-(gly)q (SEQ ID NO: 3), wherein the subscripts p and q are each independently an integer of from 0 to 20; and a pharmaceutically acceptable diluent for injection; wherein the positively charged carrier is non-covalently associated with the botulinum toxin, botulinum toxin complex, or reduced botulinum toxin complex component; and wherein the composition provides a therapeutic effect that endures for at least 16 to 24 or 16 to 36 weeks following a treatment of an individual with an effective dose of the injectable composition.

In some embodiments of these above methods and composition, the composition comprises botulinum toxin of serotype A, preferably a serotype A botulinum toxin having a molecular weight of 150 kDa. In an embodiment, the positively charged carrier has the amino acid sequence RKKRRQRRRG-(K)₁₅-GRKKRRQRRR (SEQ ID NO: 4). In an embodiment, and specifically including RKKRRQRRRG-(K)₁₅-GRKKRRQRRR (SEQ ID NO: 4), the botulinum toxin, specifically including the 150 kDa botulinum toxin A, is present in the composition in a dosage amount from more than 250 U to 500 U, 500-750 U, or 750-1000 U. In an embodiment, the botulinum toxin is present in the composition in a dosage amount selected from the group consisting of 250 U, 375 U, 500 U, 600 U, 700 U, 800 U, 900 U, and 1000 U. In an embodiment, the composition reduces the symptoms of ULS in an individual who has undergone a single treatment by injection of the composition. In certain embodiments, the duration of the treatment effect is at least 16 weeks, 20 weeks, 24 weeks.

In another of its aspects, the invention provides a method of treating an individual suffering from one or more types of ULS who is in need of treatment with injectable botulinum toxin, in which the method of treatment comprises at least one treatment course, but may encompass a treatment course having multiple treatment treatments separated by treatment intervals with prolonged duration of effect and duration time between each treatment interval, the treatment course comprising: administering by injection an initial treatment dose of a sterile injectable composition into an area of the individual in need of treatment to achieve a therapeutic effect following the initial treatment with the composition; wherein the composition comprises a botulinum toxin, a botulinum toxin complex, or a reduced botulinum toxin complex component and a positively charged carrier component comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RKKRRQRRR-(gly)q (SEQ ID NO: 3), wherein the subscripts p and q are each independently an integer of from 0 to 20; and a pharmaceutically acceptable diluent suitable for injection; wherein the botulinum toxin, botulinum toxin complex, or reduced botulinum toxin complex component is administered to the individual in a treatment dose of from more than 250 U, 250 U to 500 U, 500-750 U, or 750-1000 U, wherein the positively charged carrier is non-covalently associated with the botulinum toxin, botulinum toxin complex, or reduced botulinum toxin complex component; wherein the initial treatment dose of the composition administered by injection to the individual provides a therapeutic duration of effect lasting through at least about 16 months to at least about 24 weeks; and administering subsequent treatment doses of the composition by injection to the individual at treatment intervals comprising a duration of greater than or equal to 16 weeks to at least about 24 weeks following the initial treatment dose and between each subsequent treatment dose. Such an embodiment specifically contemplates the 150 kDa botulinum toxin type A with using the positively charged carrier RKKRRQRRRG-(K)₁₅-GRKKRRQRRR (SEQ ID NO: 4.

### DESCRIPTION OF THE FIGURES

FIG. 1 illustrates the various types of ULS.

FIG. 2 shows the change from baseline in suprahypertonic muscle group modified Ashworth score (MAS) and physician global impression of change (PGIC) at week 6.

FIG. 3 shows the mean change from baseline on suprahypertonic muscle group (SMG) modified Ashworth score (MAS) over time.

FIG. 4 shows the change in modified Ashworth score (MAS) at week 6 for suprahypertonic muscle group (SMG), wrist, fingers and elbow.

FIG. 5 shows suprahypertonic muscle group (SMG) selection by treatment group.

FIG. 6 shows the mean change from baseline on modified Ashworth score (MAS) over time for wrist.

FIG. 7 shows the mean change from baseline on modified Ashworth score (MAS) over time for fingers.

FIG. 8 shows the mean change from baseline on modified Ashworth score (MAS) over time for fingers.

FIG. 9 shows the time to loss of improvement in suprahypertonic muscle group (SMG) (modified Ashworth score (MAS) change from baseline <1 point) and physician global impression of change (PGIC)≤ or requesting retreatment.

FIG. 10 shows the proportion of subjects not requiring retreatment at weeks 12, 16, 20, 24, and 36 for an embodiment of composition of the present disclosure and described in the Examples, referred to herein as DAXI, compared to Dysport®.

FIGS. 11a and 11b show a comparison of the improvement on the MAS and the PGIC from baseline between Dysport®, Xeomin®, and DAXI.

### DETAILED DESCRIPTION OF THE INVENTION

This invention relates to injectable compositions comprising botulinum toxin, a botulinum toxin complex, or a reduced botulinum toxin complex used to treat ULS. In preferred embodiments, the compositions stabilize the toxin or enable the transport or delivery of toxin through tissues after injection such that the toxin has a longer duration of clinical efficacy compared to conventional commercial botulinum toxin complexes that are bound to exogenous albumin (e.g., BOTOX® or MYOBLOC®). The compositions of the invention may be used as injectable applications for providing a botulinum toxin to a subject, for various therapeutic, purposes, as described herein. The compositions of the invention also has an improved safety profile over other compositions and methods of delivery of botulinum toxin. In addition, these compositions can afford beneficial reductions in immune responses to the botulinum toxin. In embodiments, the injectable compositions of the invention for use in treating ULS provide long lasting efficacy, e.g., an effect lasting at least 16 weeks, 18 weeks, 20 weeks, 18, weeks, or 24 weeks, at least 6 months, or greater than 6 months, in subjects to whom such compositions, particularly those comprising botulinum toxin in amounts of 250 U to 1000 U or more, are administered by injection for the treatment of ULS.

The term “botulinum toxin” as used herein may refer to any of the known types of botulinum toxin (e.g., 150 kD botulinum toxin protein molecules associated with the different serotypes of C. botulinum), whether produced by the bacterium or by recombinant techniques, as well as any such types that may be subsequently discovered including newly discovered serotypes, and engineered variants or fusion proteins. As mentioned above, currently seven immunologically distinct botulinum neurotoxins have been characterized, namely botulinum neurotoxin serotypes A, B, C, D, E, F and G, each of which is distinguished by neutralization with type-specific antibodies. The botulinum toxin serotypes are commercially available, for example, from Sigma-Aldrich (St. Louis, MO) and from Metabiologics, Inc. (Madison, WI), as well as from other sources. The different serotypes of botulinum toxin vary in the animal species that they affect and in the severity and duration of the paralysis they evoke. At least two types of botulinum toxin, types A and B, are available commercially in formulations for treatment of certain conditions. Type A, for example, is contained in preparations of Allergan having the trademark BOTOX® and of Ipsen having the trademark DYSPORT®, and type B is contained in preparations of Elan having the trademark MYOBLOC®. A method for isolating and purifying the 150 kD botulinum toxin from the native accessory proteins is described in U.S. Pat. No. 9,469,849 which is hereby incorporated by reference in its entirety.

The term “botulinum toxin” used in the compositions of this invention can alternatively refer to a botulinum toxin derivative, that is, a compound that has botulinum toxin activity but contains one or more chemical or functional alterations on any part or on any amino acid chain relative to naturally occurring or recombinant native botulinum toxins. For instance, the botulinum toxin may be a modified neurotoxin that is a neurotoxin which has at least one of its amino acids deleted, modified or replaced, as compared to a native form, or the modified neurotoxin can be a recombinantly produced neurotoxin or a derivative or fragment thereof. For instance, the botulinum toxin may be one that has been modified in a way that, for instance, enhances its properties or decreases undesirable side effects, but that still retains the desired botulinum toxin activity. Alternatively the botulinum toxin used in this invention may be a toxin prepared using recombinant or synthetic chemical techniques, e.g. a recombinant peptide, a fusion protein, or a hybrid neurotoxin, for example prepared from subunits or domains of different botulinum toxin serotypes (See, U.S. Pat. No. 6,444,209, for instance). The botulinum toxin may also be a portion of the overall molecule that has been shown to possess the necessary botulinum toxin activity, and in such case may be used per se or as part of a combination or conjugate molecule, for instance a fusion protein. Alternatively, the botulinum toxin may be in the form of a botulinum toxin precursor, which may itself be non-toxic, for instance a non-toxic zinc protease that becomes toxic on proteolytic cleavage.

The term “botulinum toxin complex” or “toxin complex” as used herein refers to the approximately 150 kD botulinum toxin protein molecule (belonging to any one of botulinum toxin serotypes A-G), along with associated endogenous non-toxin proteins (i.e., hemagglutinin protein and non-toxin non-hemagglutinin protein produced by Clostridium botulinum bacteria). Note, however, that the botulinum toxin complex need not be derived from Clostridium botulinum bacteria as one unitary toxin complex. For example, botulinum toxin or modified botulinum toxin may be recombinantly prepared first and then subsequently combined with the non-toxin proteins. Recombinant botulinum toxin can also be purchased (e.g., from List Biological Laboratories, Campbell, CA) and then combined with non-toxin proteins.

This invention also contemplates modulation of the stability of botulinum toxin molecules through the addition of one or more exogenous stabilizers, the removal of endogenous stabilizers, or a combination thereof. For example, this invention contemplates the use of “reduced botulinum toxin complexes”, in which the botulinum toxin complexes have reduced amounts of non-toxin protein compared to the amounts naturally found in botulinum toxin complexes produced by Clostridium botulinum bacteria. In one embodiment, reduced botulinum toxin complexes are prepared using any conventional protein separation method to extract a fraction of the hemagglutinin protein or non-toxin non-hemagglutinin protein from botulinum toxin complexes derived from Clostridium botulinum bacteria. For example, reduced botulinum toxin complexes may be produced by dissociating botulinum toxin complexes through exposure to red blood cells at a pH of 7.3 (e.g., see EP 1514556 A1, hereby incorporated herein by reference). HPLC, dialysis, columns, centrifugation, and other methods for extracting proteins from proteins can be used. Alternatively, when the reduced botulinum toxin complexes are to be produced by combining synthetically produced botulinum toxin with non-toxin proteins, one may simply add less hemagglutinin or non-toxin, non-hemagglutinin protein to the mixture than what would be present for naturally occurring botulinum toxin complexes. Any of the non-toxin proteins (e.g., hemagglutinin protein or non-toxin non-hemagglutinin protein or both) in the reduced botulinum toxin complexes according to the invention may be reduced independently by any amount. In certain exemplary embodiments, one or more non-toxin proteins are reduced by at least about 0.5%, 1%, 3%, 5%, 10%, 20%, 30%, 40%, 50%, 60%, 70%, 80%, 90% or 100% compared to the amounts normally found in botulinum toxin complexes. As noted above, Clostridium botulinum bacteria produce seven different serotypes of toxin and commercial preparations are manufactured with different relative amounts of non-toxin proteins (i.e. different amount of toxin complexes). For example, MYOBLOC™ has 5000 U of Botulinum toxin type B per ml with 0.05% human serum albumin, 0.01 M sodium succinate, and 0.1 M sodium chloride. DYSPORT™ has 500 U of botulinum toxin type A-hemagglutinin complex with 125 mcg albumin and 2.4 mg lactose. In certain embodiments, substantially all of the non-toxin protein (e.g., greater than 95%, 96%, 97%, 98% or 99% of the hemagglutinin protein and non-toxin non-hemagglutinin protein) that would normally be found in botulinum toxin complexes derived from Clostridium botulinum bacteria is removed from the botulinum toxin complex. Furthermore, although the amount endogenous non-toxin proteins may be reduced by the same amount in some cases, this invention also contemplates reducing each of the endogenous non-toxin proteins by different amounts, as well as reducing at least one of the endogenous non-toxin proteins, but not the others.

As noted above, an exogenous stabilizer (e.g., albumin) is typically added to stabilize botulinum toxin formulations. For instance, in the case of BOTOX®, 0.5 mg of human albumin per 100 U of type A botulinum toxin complex to stabilize the complex. Generally, the amount of exogenous stabilizer that may be added to stabilize the compositions according to the invention is not particularly limited. In some embodiments, the amount of added stabilizer may be less than the amount conventionally added, owing to the ability of positively charged carriers of the invention to act as a stabilizer in its own right. For instance, the amount of added exogenous albumin can be any amount less than the conventional thousand-fold excess of exogenous albumin and, in certain exemplary embodiments of the invention, is only about 0.25, 0.20, 0.15, 0.10, 0.01, 0.005, 0.001, 0.0005, 0.00001, 0.000005, 0.000001, or 0.0000001 mg per 100 U of botulinum toxin. In one embodiment, no exogenous albumin is added as a stabilizer to the compositions of the invention, thus producing albumin-free botulinum toxin compositions.

A preferred composition of the invention is a liquid, botulinum toxin-containing composition that is stabilized without a proteinaceous excipient, especially without any animal protein-derived excipients. Such a liquid composition comprises a botulinum toxin, preferably botulinum toxin of serotype A, a positively charged carrier (e.g., peptide), a non-reducing disaccharide or a non-reducing trisaccharide, a non-ionic surfactant, and a physiologically compatible buffer for maintaining the pH between 4.5 and 7.5. The concentration of the non-reducing sugar in the liquid composition is in the range of 10% through 40% (w/v) and the concentration of the non-ionic surfactant is in the range of 0.005% through 0.5% (w/v). The composition of the present disclosure provides a long duration effect after treatment by a single injection. In embodiments, the botulinum toxin A has a molecular weight (MW) of 150 kDa. The composition comprises botulinum toxin, preferably botulinum toxin A, more preferably, of 150 kDa MW, a positively charged carrier (e.g., peptide) as described herein, a non-reducing disaccharide, such as sucrose, a non-ionic surfactant, such as polysorbate 20, polysorbate 40, polysorbate 60, polysorbate 80, or a sorbitan ester, and a physiologically compatible buffer, such as citric acid, acetic acid, succinic acid, tartaric acid, maleic acid, and histidine; and has a pH in the range of pH 4.5 to pH 7.5.

According to the present invention, a positively charged carrier molecule having protein transduction domains or efficiency groups, as described herein, has been found suitable as a transport system for a botulinum toxin, enabling toxin to be injected with improved penetration to target structures such as muscles. The transport occurs without covalent modification of the botulinum toxin. Besides enhancing penetration of botulinum toxin, the positively charged carriers of the invention may, in certain preferred embodiments, stabilize the botulinum toxin against degradation. In such embodiments, the hemagglutinin protein and non-toxin, non-hemagglutinin protein that are normally present to stabilize the botulinum toxin may be reduced or omitted entirely. Similarly, the exogenous albumin that is normally added during manufacturing may be omitted.

By the use of the terms “positively charged” or “cationic” in connection with the term “carrier”, it is meant that the carrier has a positive charge under at least some solution-phase conditions, more preferably, under at least some physiologically compatible conditions. More specifically, “positively charged” and “cationic” as used herein, means that the group in question contains functionalities that are charged under all pH conditions, for instance, a quaternary amine, or contains a functionality which can acquire positive charge under certain solution-phase conditions, such as pH changes in the case of primary amines. More preferably, “positively charged” or “cationic” as used herein refers to those groups that have the behavior of associating with anions over physiologically compatible conditions. Polymers with a multiplicity of positively-charged moieties need not be homopolymers, as will be apparent to one skilled in the art. Other examples of positively charged moieties are well known in the prior art and can be employed readily, as will be apparent to those skilled in the art.

Generally, the positively-charged carrier (also referred to as a “positively charged backbone”) is typically a chain of atoms, either with groups in the chain carrying a positive charge at physiological pH, or with groups carrying a positive charge attached to side chains extending from the backbone. In certain preferred embodiments, the positively charged backbone is a cationic peptide. As used herein, the term “peptide” refers to an amino acid sequence, but carries no connotation with respect to the number of amino acid residues within the amino acid sequence. Accordingly, the term “peptide” may also encompass polypeptides and proteins. In certain preferred embodiments, the positively charged backbone itself will not have a defined enzymatic or therapeutic biologic activity. In certain embodiments, the backbone is a linear hydrocarbon backbone which is, in some embodiments, interrupted by heteroatoms selected from nitrogen, oxygen, sulfur, silicon and phosphorus. The majority of backbone chain atoms are usually carbon. Additionally, the backbone will often be a polymer of repeating units (e.g., amino acids, poly(ethyleneoxy), poly(propyleneamine), polyalkyleneimine, and the like) but can be a heteropolymer. In one group of embodiments, the positively charged backbone is a polypropyleneamine wherein a number of the amine nitrogen atoms are present as ammonium groups (tetra-substituted) carrying a positive charge. In another embodiment, the positively charged backbone is a nonpeptidyl polymer, which may be a hetero- or homo-polymer such as a polyalkyleneimine, for example a polyethyleneimine or polypropyleneimine, having a molecular weight of from about 10,000 to about 2,500,000, preferably from about 100,000 to about 1,800,000, and most preferably from about 500,000 to about 1,400,000. In another group of embodiments, the backbone has attached a plurality of side-chain moieties that include positively charged groups (e.g., ammonium groups, pyridinium groups, phosphonium groups, sulfonium groups, guanidinium groups, or amidinium groups). The sidechain moieties in this group of embodiments can be placed at spacings along the backbone that are consistent in separations or variable. Additionally, the length of the sidechains can be similar or dissimilar. For example, in one group of embodiments, the sidechains can be linear or branched hydrocarbon chains having from one to twenty carbon atoms and terminating at the distal end (away from the backbone) in one of the above-noted positively charged groups. The association between the positively charged carrier and the botulinum toxin is by non-covalent interaction, non-limiting examples of which include ionic interactions, hydrogen bonding, van der Waals forces, or combinations thereof. Examples of positively charged backbones and efficiency groups for use of the invention are described in U.S. Pat. Nos. 8,623,811, 9,956,4435, and 9,211,248, which are hereby incorporated by reference in their entirety.

In one group of embodiments, the positively charged backbone is a polypeptide having multiple positively charged sidechain groups (e.g., lysine, arginine, ornithine, homoarginine, and the like). Preferably, the polypeptide has a molecular weight from about 100 to about 1,500,000, more preferably from about 500 to about 1,200,000, most preferably from about 1000 to about 1,000,000. One of skill in the art will appreciate that when amino acids are used in this portion of the invention, the sidechains can have either the D- or L-form (R or S configuration) at the center of attachment. In certain preferred embodiments, the polypeptide has a molecular weight from about 500 to about 5000, more preferably from 1000 to about 4000, more preferably from 2000 to about 3000. In other preferred embodiments, the polypeptide comprises 10 to 20 amino acids, or 10, 11, 12, 13, 14, 15, 16, 17, 18, 19, or 20 amino acids, preferably polylysine.

Alternatively, the backbone may comprise amino acid analogs and/or synthetic amino acids. The backbone may also be an analog of a polypeptide such as a peptoid. See, for example, Kessler, Angew. Chem. Int. Ed. Engl. 32:543 (1993); Zuckermann et al. Chemtracts-Macromol. Chem. 4:80 (1992); and Simon et al. Proc. Nat'l. Acad. Sci. USA 89:9367 (1992)). Briefly, a peptoid is a polyglycine in which the sidechain is attached to the backbone nitrogen atoms rather than the α-carbon atoms. As above, a portion of the sidechains will typically terminate in a positively charged group to provide a positively charged backbone component. Synthesis of peptoids is described in, for example, U.S. Pat. No. 5,877,278, which is hereby incorporated by reference in its entirety. As the term is used herein, positively charged backbones that have a peptoid backbone construction are considered “non-peptide” as they are not composed of amino acids having naturally occurring sidechains at the alpha-carbon locations.

A variety of other backbones can be used employing, for example, steric or electronic mimics of polypeptides wherein the amide linkages of the peptide are replaced with surrogates such as ester linkages, thioamides (—CSNH—), reversed thioamide (—NHCS—), aminomethylene (—NHCH₂—) or the reversed methyleneamino (—CH₂NH—) groups, keto-methylene (—COCH₂—) groups, phosphinate (—PO₂RCH₂—), phosphonamidate and phosphonamidate ester (—PO₂RNH—), reverse peptide (—NHCO—), trans-alkene (—CR═CH—), fluoroalkene (—CF═CH—), dimethylene (—CH₂CH₂—), thioether (—CH₂S—), hydroxyethylene (—CH(OH)CH₂—), methyleneoxy (—CH₂O—), tetrazole (CN₄), sulfonamido (—SO₂NH—), methylenesulfonamido (—CHRSO₂NH—), reversed sulfonamide (—NHSO₂—), and backbones with malonate and/or gem-diamino-alkyl subunits, for example, as reviewed by Fletcher et al. ((1998) Chem. Rev. 98:763) and detailed by references cited therein. Many of the foregoing substitutions result in approximately isosteric polymer backbones relative to backbones formed from «-amino acids.

In each of the backbones provided above, sidechain groups can be appended that carry a positively charged group. For example, the sulfonamide-linked backbones (—SO₂NH— and —NHSO₂—) can have sidechain groups attached to the nitrogen atoms. Similarly, the hydroxyethylene (—CH(OH)CH₂—) linkage can bear a sidechain group attached to the hydroxy substituent. One of skill in the art can readily adapt the other linkage chemistries to provide positively charged sidechain groups using standard synthetic methods.

In one embodiment, the positively charged backbone is a polypeptide having protein transduction domains (also referred to as efficiency groups). As used herein, an efficiency group or protein transduction domain is any agent that has the effect of promoting the translocation of the positively charged backbone through a tissue or cell membrane. Non-limiting examples of protein transduction domains or efficiency groups include-(gly) n1-(arg) n2 (SEQ ID NO: 5), HIV-TAT or fragments thereof, or the protein transduction domain (PTD) of Antennapedia, or a fragment thereof, in which the subscript n1 is an integer of from 0 to 20, more preferably 0 to 8, still more preferably 2 to 5, and the subscript n2 is independently an odd integer of from about 5 to about 25, more preferably about 7 to about 17, most preferably about 7 to about 13. In some embodiments, the HIV-TAT fragment does not contain the cysteine-rich region of the HIV-TAT molecule, in order to minimize the problems associated with disulfide aggregation. Preferably, the fragments of the HIV-TAT and Antennapedia protein transduction domains retain the protein transduction activity of the full protein. Still further preferred are those embodiments in which the HIV-TAT fragment has the amino acid sequence (gly)ₚ-RGRDDRRQRRR-(gly)_{q} (SEQ ID NO: 1), (gly)ₚ-YGRKKRRQRRR-(gly)_{q} (SEQ ID NO: 2) or (gly)ₚ-RKKRRQRRR-(gly)_{q} (SEQ ID NO:3) wherein the subscripts p and q are each independently an integer of from 0 to 20, or wherein p and q are each independently the integer 1. In another embodiment, the fragment or efficiency group is attached to the backbone via either the C-terminus or the N-terminus of the fragment or amino acid sequence of the efficiency group. In certain preferred embodiments, p is one and q is zero or p is zero and q is one. Preferred HIV-TAT fragments are those in which the subscripts p and q are each independently integers of from 0 to 8, more preferably 0 to 5. In another preferred embodiment the positively charged side chain or branching group is the Antennapedia (Antp) protein transduction domain (PTD), or a fragment thereof that retains activity. These are known in the art, for instance, from Console et al., J. Biol. Chem. 278:35109 (2003) and a non-limiting example of an Antennapedia PTD contemplated by this invention is the PTD having the amino acid sequence SGRQIKIWFQNRRMKWKKC (SEQ ID NO: 6). In other embodiments, the positively charged carrier is a positively charged peptide having the amino acid sequence RKKRRQRRR-G-(K)₁₅-G-RKKRRQRRR (SEQ ID NO: 4); or a positively charged peptide having the amino acid sequence YGRKKRRQRRR-G-(K)₁₅-G-YGRKKRRQRRR (SEQ ID NO: 7); or a positively charged peptide having the amino acid sequences RGRDDRRQRRR-G-(K)₁₅-G-RGRDDRRQRRR (SEQ ID NO: 8) for use in the compositions and methods of the invention.

Preferably the positively charged carrier includes side-chain positively charged protein transduction domains or positively charged efficiency groups in an amount of at least about 0.01%, as a percentage of the total carrier weight, preferably from about 0.01 to about 50 weight percent, more preferably from about 0.05 to about 45 weight percent, and most preferably from about 0.1 to about 30 weight %. For positively charged protein transduction domains having the formula-(gly)ₙ₁-(arg)ₙ₂ (SEQ ID NO: 5), a preferred range is from about 0.1 to about 25%.

In another embodiment, the backbone portion is a polylysine and positively charged protein transduction domains are attached to the lysine sidechain amino groups or to the C- or N termini. In some preferred embodiments, the polylysine may have a molecular weight that is at least 100, 200, 300, 400, 500, 600, 700, 800, 900, 1000, 1500, 2000, 2500, 3000, 3500, 4000, 4500, 5000, 5500, or 6000 D, and less than about 2,000,000, 1,000,000, 500,000, 250,000, 100,000, 75,000, 50,000, and 25,000 D. Within the range of 100 to 2,000,000 D, it is contemplated that the lower and/or upper range may be increased or decreased, respectively, by 100, with each resulting sub-range being a specifically contemplated embodiment of the invention. In some exemplary embodiments, the polylysine has a molecular weight from about 1,000 to about 1,500,000 D, from about 2,000 to about 800,000 D, or from about 3,000 to about 200,000 D. In other exemplary embodiments, the polylysine has molecular weight from about 100 to about 10,000 D, from about 500 to about 5,000 D, from about 1,000 to about 4,000 D, from about 1,500 to about 3,500 D or from about 2,000 to about 3,000 D. Preferred is a polylysine polypeptide having 10 to 20 lysines (SEQ ID NO: 9), more preferably, 15 lysines. In some embodiments, the polylysine contemplated by this invention can be any of the commercially available (Sigma Chemical Company, St. Louis, Mo., USA) polylysines such as, for example, polylysine having MW>70,000, polylysine having MW of 70,000 to 150,000, polylysine having MW 150,000 to 300,000 and polylysine having MW>300,000. The selection of an appropriate polylysine will depend on the remaining components of the composition and will be sufficient to provide an overall net positive charge to the composition and provide a length that is preferably from one to four times the combined length of the negatively charged components. Preferred positively charged protein transduction domains or efficiency groups include, for example,-gly-gly-gly-arg-arg-arg-arg-arg-arg-arg (-Gly₃Arg₇ (SEQ ID NO: 10)) or HIV-TAT.

In another preferred embodiment the positively charged backbone is a polyalkyleneimine, non-limiting examples of which include polyethyleneimine, polypropyleneimine, and polybutyleneimine. In certain embodiments, the polyalkyleneimine has a molecular weight of at least 100, 200, 300, 400, 500, 600, 700, 800, 900, 1000, 1500, 2000, 2500, 3000, 3500, 4000, 4500, 5000, 5500, or 6000 D, and less than about 2,000,000, 1,000,000, 500,000, 250,000, 100,000, 75,000, 50,000, and 25,000 D. Within the range of 100 to 2,000,000 D, it is contemplated that the lower and/or upper range may be increased or decreased, respectively, by 100, with each resulting sub-range being a specifically contemplated embodiment of the invention.

In other embodiments of this invention, the carrier is a relatively short polylysine or polyethyleneimine (PEI) backbone (which may be linear or branched) and which has positively charged branching groups. Without wishing to be constrained by theory, it is believed that such carriers are useful for minimizing uncontrolled aggregation of the backbones and botulinum toxin in a therapeutic composition, which causes the transport efficiency to decrease dramatically. When the carrier is a relatively short linear polylysine or PEI backbone, the backbone will have a molecular weight of less than 75,000 D, more preferably less than 30,000 D, and most preferably, less than 25,000 D. When the carrier is a relatively short branched polylysine or PEI backbone, however, the backbone will have a molecular weight less than 60,000 D, more preferably less than 55,000 D, and most preferably less than 50,000 D.

In one particularly interesting embodiment, the non-native molecules are cationic peptides that have no inherent botulinum-toxin-like activity and that also contain one or more protein transduction domains as described herein. Without wishing to be bound by any particular scientific theory, it is believed that the peptides enhance tissue penetration of molecules associated in complex after injection, while enhancing stabilization of the botulinum toxin in skin and in vitro. It is believed that the enhanced tissue penetration afforded by these peptides in particular affords reduced antigenicity, a better safety profile, enhanced potency, faster onset of clinical efficacy or longer duration of clinical efficacy compared to conventional commercial botulinum toxin complexes that are bound to exogenous albumin (e.g., BOTOX® or MYOBLOC®).

In preferred embodiments, the concentration of positively charged carriers in the compositions according to the invention is sufficient to enhance the delivery of the botulinum toxin to molecular targets such as, for example, motor nerve plates. Furthermore, without wishing to be bound by theory, it is believed that the penetration rate follows receptor-mediated kinetics, such that tissue penetration increases with increasing amounts of penetration-enhancing-molecules up to a saturation point, upon which the transport rate becomes constant. Thus, in a preferred embodiment, the amount of added penetration-enhancing-molecules is equal to the amount that maximizes penetration rate right before saturation. A useful concentration range for the positively charged carrier (or carrier peptide) in the injectable compositions of this invention is about 0.1 pg of carrier per Unit (U) of botulinum toxin (0.1 pg/U) to about 1.0 mg per Unit (mg/U) of the botulinum toxin as described herein. A useful concentration range for the positively charged carrier (or carrier peptide) in the topical compositions of the invention is about 1.0 pg/U to 0.5 mg/U of botulinum toxin (amount of carrier/U of botulinum toxin). In other embodiments, the positively charged carrier (or carrier peptide) is present in the injectable compositions of the invention in the range of, for example, 10 ng/U to 200 ng/U of botulinum toxin, or in the range of 1 ng/U to 1000 ng/U of botulinum toxin; or in the range of 0.1 ng/U to 10,000 ng/U of botulinum toxin. In some embodiments, the amount of positively charged carrier (or carrier peptide) to Units of botulinum toxin present in the compositions of the invention is, by way of nonlimiting example, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, etc. ng of carrier per Unit of botulinum toxin (ng/U). Preferably, the botulinum toxin is of serotype A, and particularly, the 150 kD form of serotype A botulinum toxin.

In general, methods and procedures for measuring the activity of botulinum toxin, i.e., units (U) of botulinum toxin activity, are known to and practiced by those having skill in the art. Briefly, median lethality assays (LD₅₀ assays) in mice are conventionally used to estimate the number of units of botulinum toxin with a high degree of precision. Doses of all commercially available botulinum toxins are expressed in terms of units of biologic activity. By way of example, one unit of botulinum toxin corresponds to the calculated median intraperitoneal lethal dose (LD50) in female Swiss-Webster mice. See, Hoffman, R. O. et al., 1986, Int. Ophthalmol. Clin., 26:241-50, as well as DePass, L. R., 1989, Toxicol. Letters, 49:159-170; and Pearce, L. B. et al., 1994, Toxicol. Appl. Pharmacol., 128:69-77, which also describe lethality assays in the art. More particularly, a suitable method for determining botulinum toxin units for a botulinum toxin component of the compositions of the invention is as follows: Forty-eight (48) female CD-1 mice weighing 17-23 grams are randomly assigned to six doses of the test article (1.54, 1.31, 1.11, 0.95, 0.80, and 0.68 U/0.5 mL), eight (8) animals per dose group. The test article refers to the botulinum toxin preparation or sample being assayed or tested. The animals are housed eight per cage and are weighed within 24 hours of dosing with the test article. On the day of dosing, the test article is diluted to the appropriate concentrations in isotonic saline (0.9% NaCl). Each animal is administered 0.5 mL of diluted test article via intraperitoneal injection. After injection, mice are returned to the cage and fatalities are recorded daily for three days. Lethality is scored 72 hours post injection and the results are analyzed by probit or logistic analysis to derive the LD₅₀ value relative to a reference standard that is assessed using the same dosing regimen. By way of example, the reference standard is a specifically qualified and calibrated lot of the same composition of the invention that is used for comparison to derive relative potency of the test article. The determined LD₅₀ value is then corrected for the cumulative dilutions performed to assign a relative potency value for the neat (undiluted) test article.

Compositions of this invention are preferably in a form that permits injection into the affected muscles of subjects or patients. The term “in need” is meant to include both pharmaceutical or health-related needs (e.g., treating conditions involving undesirable dystonic contractions or muscle spasms). In preferred embodiments, the compositions are prepared by mixing the botulinum toxin (either containing the associated non-toxin proteins or reduced associated non-toxin proteins) with the positively charged carrier, and usually with one or more additional pharmaceutically acceptable carriers or excipients. In their simplest form, they may contain an aqueous pharmaceutically acceptable diluent, such as buffered saline (e.g., phosphate buffered saline). However, the compositions may contain other ingredients typically found in injectable pharmaceutical or cosmeceutical compositions, including a pharmaceutically acceptable carrier, vehicle or medium that is compatible with the tissues to which it will be applied. The term “pharmaceutically acceptable,” as used herein, means that the compositions or components thereof so described are suitable for use in contact with these tissues or for use in patients in general without undue toxicity, incompatibility, instability, allergic response, and the like. As appropriate, compositions of the invention may comprise any ingredient conventionally used in the fields under consideration.

In terms of their form, compositions of this invention may include solutions, emulsions (including microemulsions), suspensions, gels, powders, or other typical solid or liquid compositions used for injection to muscle. In preferred embodiments, the compositions of the invention are present in low-viscosity, sterile formulations suitable for injection with a syringe. As used herein, the terms compositions and formulations are essentially interchangeable when referring to the compositions and formulations according to the present invention. The compositions of the invention may be in the form of a lyophilized powder that is reconstituted using a pharmaceutically acceptable liquid diluent prior to injection. In certain embodiments, the lyophilized powder is reconstituted with a liquid diluent to form an injectable formulation with a viscosity of about 0.1 to about 2000 cP, more preferably about 0.2 to about 500 cP, even more preferably about 0.3 to about 50 cP, and even more preferably about 0.4 to about 2.0 cP. The compositions of the invention may contain, in addition to the botulinum toxin and positively charged carrier, other ingredients typically used in such products, such as antimicrobials, hydration agents, tissue bulking agents or tissue fillers, preservatives, emulsifiers, natural or synthetic oils, solvents, surfactants, detergents, gelling agents, antioxidants, fillers, thickeners, powders, viscosity-controlling agents and water, and optionally including anesthetics, anti-itch actives, botanical extracts, conditioning agents, minerals, polyphenols, silicones or derivatives thereof, vitamins, and phytomedicinals.

The injectable compositions according to this invention may be in the form of controlled-release or sustained-release compositions which comprise botulinum toxin and positively charged carrier encapsulated or otherwise contained within a material such that they are released within the tissue in a controlled manner over time. The composition comprising the botulinum toxin and positively charged carrier may be contained within matrixes, liposomes, vesicles, microcapsules, microspheres and the like, or within a solid particulate material, all of which is selected and/or constructed to provide release of the botulinum toxin over time. The botulinum toxin and the positively charged carrier may be encapsulated together (i.e., in the same capsule) or separately (i.e., in separate capsules).

In embodiments, compositions of the invention comprise liquid (aqueous) compositions (or formulations) comprising a botulinum toxin as described herein, a positively charged carrier (or peptide) as described herein, a non-reducing disaccharide or a non-reducing trisaccharide, a non-ionic surfactant, and a physiologically compatible buffer, which is capable of maintaining a suitable pH, such as a pH in the range of pH 4.5 to pH 7.5, or pH 4.5 to pH 6.8, or pH 4.5 to pH 6.5. It is to be understood that a suitable pH also includes the upper and lower pH values in the range, e.g., a pH of 6.5 or a pH of 7.5. The concentration of the non-reducing sugar in the liquid composition is in the range of 10% through 40% (w/v) and the concentration of the non-ionic surfactant is in the range of 0.005% through 0.5% (w/v). The liquid compositions may be dried, preferably by lyophilization, to produce stabilized solid compositions, which may thereafter be reconstituted for use, for example, using sterile saline or other known physiologically and pharmaceutically acceptable diluents, excipients, or vehicles, especially those known for use in injectable formulations. Preferably, the dried, e.g., lyophilized, solid compositions are noncrystalline and amorphous solid compositions, and may be in the form of powders, for example. Also, preferably, the compositions of the invention do not include animal protein-derived products, such as albumin. Compositions that are suitable for the invention are also described in U.S. Pat. No. 9,340,587 B2, the entire contents of which are incorporated herein by reference. In particular embodiments the compositions comprise botulinum toxin of serotype A. In other particular embodiments, the compositions comprise botulinum toxin of serotype A which has a molecular weight of 150 kDa.

In certain embodiments, the compositions of the present disclosure contain a non-reducing sugar, which is preferably a disaccharide, non-limiting examples of which include trehalose, including its anhydrous and hydrated forms, or sucrose, as well as combinations thereof. In some embodiments, the hydrated form of trehalose, trehalose-dihydrate, is preferable. In other embodiments, the compositions contain a trisaccharide, a non-limiting example of which is raffinose. In general, the concentration of the non-reducing sugar, preferably a disaccharide, e.g., sucrose, in the compositions of the invention are in the range of 10% to 40% (w/v), preferably 10% to 25% (w/v), more preferably 15% to 20% (w/v). In some preferred embodiments, the concentration of the non-reducing sugar, preferably a disaccharide, e.g., sucrose, is 10%, 11%, 12%, 13%, 14%, 15%, 16%, 17%, 18%, 19% or 20% (w/v).

In general, the compositions of the invention may include any non-ionic surfactant that has the ability to stabilize botulinum toxin and that is suitable for pharmaceutical use. In some embodiments, the non-ionic surfactant is a polysorbate, such as, by way of nonlimiting example, polysorbate 20, polysorbate 40, polysorbate 60, and polysorbate 80. In other embodiments, the non-ionic surfactant is a sorbitan ester, non-limiting examples of which include SPAN® 20, SPAN® 60, SPAN® 65, and SPAN® 80. The non-ionic surfactants Triton® X-100 or NP-40 may also be used. In addition, a combination of the different non-ionic surfactants may be used. In certain preferred embodiments, the non-ionic surfactant is a polysorbate, a poloxamer and/or a sorbitan; polysorbates and sorbitans are particularly preferred. In embodiments, the non-ionic surfactant is present in the compositions of the invention in the range of 0.005% to 0.5%, or in the range of 0.01% to 0.2%, or in the range of 0.02% to 0.1% or in the range of 0.05 to 0.08%, inclusive of the upper and lower values. In addition, the compositions of the invention may contain a non-ionic surfactant in the amount of 0.01%, 0.02%, 0.03%, 0.04%, 0.05%, 0.06%, 0.07%, 0.08%, 0.09%, 0.10%, 0.11%, 0.12%, 0.13%, 0.14%, or 0.15%.

In general for the compositions of the invention, any physiologically compatible buffer capable of maintaining the pH in the above ranges is suitable for use. Non-limiting examples of such buffers include salts of citric acid, acetic acid, succinic acid, tartaric acid, maleic acid, and histidine. Non-limiting examples of suitable buffer concentrations include buffer concentrations in the range of 0.400% to 0.600%; 0.450% to 0.575%, or 0.500% to 0.565%. The compositions of the invention may also comprise a mixture of buffer salts, non-limiting examples of which include citrate/acetate, citrate/histidine, citrate/tartrate, maleate/histidine, or succinate/histidine. Accordingly, a composition of the invention which provides a long duration effect after treatment by a single injection includes a botulinum toxin, such as botulinum toxin A or botulinum toxin A of 150 kDa MW, as described herein, a positively charged carrier (or peptide) as described herein, a non-reducing disaccharide, such as sucrose, a non-ionic surfactant, such as polysorbate 20, polysorbate 40, polysorbate 60, polysorbate 80, or a sorbitan ester, and a physiologically compatible buffer, such as citric acid, acetic acid, succinic acid, tartaric acid, maleic acid, and histidine, which is capable of maintaining a suitable pH, such as a pH in the range of pH 4.5 to pH 6.5 or in the range of pH 4.5 to pH 7.5, in w/v amounts as described herein.

A particular composition of the invention is an albumin-free, liquid (aqueous) composition which comprises a botulinum toxin, preferably botulinum toxin of serotype A, or a botulinum toxin A having a molecular weight of 150 kDa; a positively charged carrier (e.g., peptide); a non-reducing disaccharide or a non-reducing trisaccharide, preferably a disaccharide, present in a range of 10% through 40% (w/v); a non-ionic surfactant, preferably, a polysorbate or sorbitan ester, present in the range of 0.005% through 0.5% (w/v); and a physiologically compatible buffer, such as citric acid, acetic acid, succinic acid, tartaric acid, maleic acid, or histidine, present in the range of 0.400% to 0.600%; 0.450% to 0.575%, or 0.500% to 0.565%, for maintaining the pH between 4.5 and 7.5.

In particularly preferred embodiments, the pharmaceutical formulation for injection comprises L-Histidine and/or L-Histidine hydrochloride as further stabilizing agents. In particularly preferred embodiments, the excipients comprise or consist of trehalose dihydrate, polysorbate 20, L-histidine and L-histidine hydrochloride.

Botulinum toxin formulations according to the invention can be delivered by injection (typically using a syringe) to muscles underlying the skin, or to glandular structures within the skin, in an effective amount to produce paralysis, produce relaxation, alleviate contractions, prevent or alleviate spasms, reduce glandular output, or other desired effects. Local delivery of the botulinum toxin in this manner could afford dosage reductions, reduce toxicity and allow more precise dosage optimization for desired effects relative to injectable or implantable materials.

The compositions of the invention are administered to deliver an effective amount, preferably a therapeutically effective amount, of the botulinum toxin. The term “effective amount” or “therapeutically effective amount” as used herein means an amount of a botulinum toxin as defined above that is sufficient to produce the desired muscular paralysis or other biological effect, but that implicitly is a safe amount, i.e., one that is low enough to avoid serious side effects.

The compositions of the invention may contain an appropriate effective amount of the botulinum toxin for application as a single-dose treatment, or may be more concentrated, either for dilution at the place of administration or for use in multiple applications and/or sequential applications over periods of time. Through the use of the positively charged carrier this invention, a botulinum toxin can be administered by injection to a subject for treating conditions such as ULS. The botulinum toxin is administered by injection to muscles.

Most preferably, the compositions are administered by or under the direction of a physician or other health care professional. They may be administered in a single treatment or in a series of treatments over time. In preferred embodiments, a composition according to the invention is injected at a location or locations where an effect associated with botulinum toxin is desired.

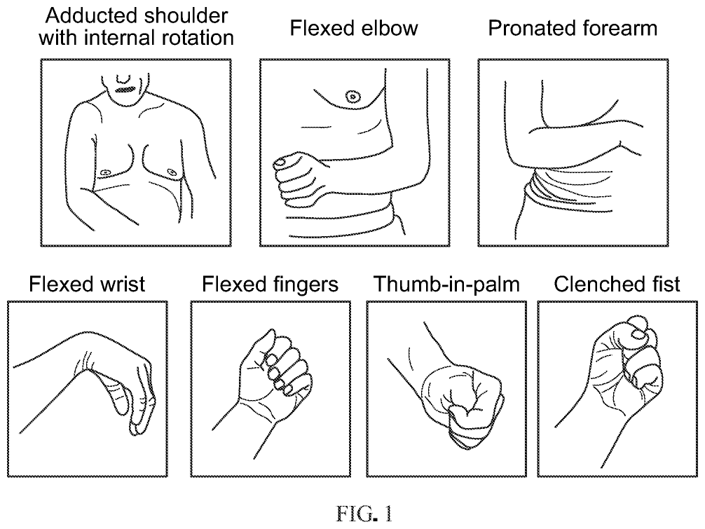

An individual with ULS can have different muscular patterns of spasticity and different regions of spasticity within the left and/or right upper limbs (also referred to as types of ULS). The types of spasticity include clenched fist, flexed fingers, flexed elbow, flexed wrist, adducted and internally rotated shoulder, pronated forearm, and thumb-in-palm. FIG. 1 illustrates different types of ULS. The muscles that can contribute to the different types of ULS of FIG. 1 are provided in Table 1 below as are dose ranges and amounts for treating each type of ULS.

TABLE 1

| | Muscle Targets for Injection, prerably those that are | Adult Dose Range of 150 |
|---|---|---|
| Type of ULS | italicized | kDa Type A BoNT |
| Clenched Fist | flexor digitorum superficialis and flexor digitorum | 50 U to 150 U, such as 50 U, |
| → Sub-type: | profundus as well as flexor pollicis brevis/opponens, | 100 U, or 150 U |
| Thumb-in-palm | flexor pollicis longus, and adductus pollicis longus | |
| | Thumb-in palm: flexor pollicis longus, adductor pollicis; | Thumb-in-palm: 25 U to 100 |
| | flexor pollicis brevis/opponens, and first dorsal interosseous | U, such as 25 U, 50 U, 75 U, |
| | as well as flexor digitorum profundus | or 100 U |
| Flexed Fingers | flexor digitorum superficialis and flexor digitorum | 50 U to 150 U, such as 50 U, |
| | profundus as well as flexor carpi radialis, flexor carpi | 100 U, or 150 |
| | ulnaris, and lumbricales | |
| Flexed Elbow | biceps; brachioradialis; and brachialis as well as pronator | 150 U to 300 U, such as 150 |
| | teres and pronator quadratus | U, 200 U, 250 U, or 300 U |
| Flexed Wrist | flexor carpi radialis and flexor carpi ulnaris as well as | 50 U to 100 U, such as 50 U, |
| | palmaris longus, flexor pollicis longus, flexor digitorum | 75 U, or 100 U |
| | superficialis, flexor digitorum profundus | |
| Adducted and | pectoralis complex and latissimus dorsi as well as teres | 50 U to 300 U, such as 50 U, |
| internally rotated | major, subscapularis, deltoid media, brachialis, levator | 100 U, 150 U, 200 U, 250 U, |
| shoulder | scapulae | or 300 U |
| Pronated forearm | pronator quadratus and pronator teres as well as flexor | 50 U to 300 U, such as 50 U, |
| | carpi ulnaris, brachialis, and brachioradialis | 100 U, 150 U, 200 U, 250 U, |
| | | or 300 U |

In embodiments, the dose injected per muscle can be as defined in Table 2 below:

| | Dose of 150 kDa | |
|---|---|---|
| Muscle | Type A BoNT | |
| Biceps brachii | 0-100 | U |
| Brachialis | 50-200 | U |
| Brachioradialis | 25-100 | U |
| Flexor carpi radialis | 50-100 | U |
| Flexor carpi ulnaris | 25-75 | U |
| Flexor digitorum profundus | 25-100 | U |
| Flexor digitorum superficialis | 20-100 | U |
| (sublimis) | | |
| Pronator teres | 45-200 | U |
| Pronator quadratus | 0-100 | U |
| Adductor Pollicis | 10-20 | U |
| Flexor pollicis longus | 40-50 | U |
| Flexor pollicis brevis/Opponens | 12.5-20 | U |
| pollicis | | |
| Pectoralis complex | 75-200 | U |
| Latissimas dorsi | 75-150 | U |

An individual can present with one or more types of ULS. In some embodiments, the individual to be treated comprises a flexed wrist and a flexed elbow, and optionally, a clenched first or flexed fingers. The clenched first can be of the type of thumb-in-palm. The individual can further presented with one or more of a pronated forearm or an adducted and internally rotated shoulder.

Because of its nature, the botulinum toxin is administered at an amount, application rate, and frequency that will produce the desired result without producing any adverse or undesired results. In embodiments, a single treatment with an effective dose of the compositions of the invention affords an effect of long duration such that during a course of treatment for an indication treatable by botulinum toxin or series of injections during a single treatment session, with a concomitant effect that endures over extended periods of time, e.g., at least 4 months, 5 months, 6 months, 7 months, or 8 months or longer. The longer duration of action provides for longer intervals or time periods between treatments where multiple treatments are used to maintain a treatment goal or effect. In an embodiment, the longer duration of effect of the composition following administration to, or dosing of, an individual with a composition of the invention providing about 250 U to 1000 U; or more specifically, from about 250 U to 500 U or from about 500 U to 600 U or from about 600 U to 700 U or from about 700 U to 800 U or from about 800 U to 900 U or from about 900 U to 1000 U, of botulinum toxin, for example, at least 16 weeks or 4 months or more, such as 16, 18, 20, 22, 24, 26, 28, 30, 32 or 36 weeks, including in between, is relative to a duration of effect of a botulinum toxin-containing composition or product that does not contain a positively charged carrier (or peptide) according to the present invention. In particular embodiments the the above doses and durations are associated with the administration of the 150 kDa type A botulinum toxin non-covalently associated with the positively charged carrier.

In certain embodiments, the compositions of the invention, which comprise a botulinum toxin and a positively charged carrier comprising a positively charged polymeric backbone with one or more covalently attached positively charged efficiency groups as described herein, are administered as a single injection to a subject or patient in need thereof in an amount or at a dose which provides about 250 U to 1000 U; or more specifically, from about 250 U to 500 U or from about 500 U to 600 U or from about 600 U to 700 U or from about 700 U to 800 U or from about 800 U to 900 U or from about 900 U to 1000 U, of botulinum toxin per treatment dose per subject for the treatment of ULS. According to the invention, a treatment effect endures for several weeks or months, for example, for at least 16 weeks, for at least 18 weeks, for at least 16 weeks, for at least 20 weeks, for at least 22 weeks, for at least 24 weeks, or for at least 4 months, 5 months, or greater than 6 months, such as 6, 7, or 8 months, or longer. In embodiments, the botulinum toxin is of serotype A, B, C, D, E, F, or G. In an embodiment, the botulinum toxin is of serotype A. In an embodiment, the serotype A botulinum toxin has a molecular weight of 150 kDa. In an embodiment, the serotype A botulinum toxin is in the form of a higher molecular weight complex as described supra. In preferred embodiments, the 150 kDa botulinum toxin or the higher molecular weight forms of the toxin are in albumin-free formulations. In an embodiment, the positively charged polymeric backbone is polylysine or polyethyleneimine. In an embodiment, the one or more positively charged efficiency groups include-(gly)ₙ₁-(arg)ₙ₂ (SEQ ID NO: 5), in which the subscript n1 is an integer of from 0 to 20, more preferably 0 to 8, still more preferably 2 to 5, and the subscript n2 is independently an odd integer of from about 5 to about 25, more preferably about 7 to about 17, most preferably about 7 to about 13. In some embodiments, the one or more positively charged efficiency groups has the amino acid sequence (gly)ₚ-RGRDDRRQRRR-(gly)_{q} (SEQ ID NO: 1), (gly)ₚ-YGRKKRRQRRR-(gly)_{q} (SEQ ID NO: 2) or (gly)ₚ-RKKRRQRRR-(gly)_{q} (SEQ ID NO: 3), wherein the subscripts p and q are each independently an integer of from 0 to 20. In certain preferred embodiments, p is one and q is zero or p is zero and q is one. In other preferred embodiments, the subscripts p and q are each independently integers of from 0 to 8, more preferably 0 to 5. In a particular embodiment, the positively charged carrier has the amino acid sequence RKKRRQRRRG-(K)₁₅-GRKKRRQRRR (SEQ ID NO: 4) or the reverse sequence thereof. In other embodiments, the one or more positively charged efficiency groups is attached to the positively charged backbone via either the C-terminus or the N-terminus of the efficiency group, e.g., amino acid sequence. In some embodiments, the one or more positively charged efficiency groups are attached to either end, or both ends, of the positively charged polylysine backbone of the positively charged carrier. In particular embodiments, the positively charged backbone is polylysine. In particular embodiments the the above doses and durations are associated with the administration of the 150 kDa type A botulinum toxin non-covalently associated with the positively charged carrier.

Without wishing to be limiting, in a course of treatment, the compositions of the invention may be administered at less frequent intervals following an initial treatment dose based on the extended duration of effect afforded by the therapeutically effective doses of the compositions and methods of the invention as described herein. For example, the compositions of the invention may be administered (or dosed) to an individual in need less than 2, 3, or 4 times per year or about every 16, 20, 22, or 24, 26, 28, 30, 32, 34 or 36 weeks, by the practice of the methods of the invention. A median duration between doses may be 16 weeks, at least 18 weeks, at least 20 weeks, at least 22 weeks, or at least 24 weeks, depending on the therapeutic treatment and/or the desire for treatment as determined by the individual being treated. A composition of the invention for treating ULS may be dosed at the appropriate interval at about 250 U to 1000 U; or more specifically, from about 250 U to 500 U or from about 500 U to 600 U or from about 600 U to 700 U or from about 700 U to 800 U or from about 800 U to 900 U or from about 900 U to 1000 U, of botulinum toxin in the composition.

This invention also contemplates the use of a variety of delivery devices for injecting botulinum toxin-containing compositions described herein across skin. Such devices may include, without limitation, a needle and syringe, or may involve more sophisticated devices capable of dispensing and monitoring the dispensing of the composition, and optionally monitoring the condition of the subject in one or more aspects (e.g., monitoring the reaction of the subject to the substances being dispensed).

In some embodiments, the compositions can be pre-formulated and/or pre-installed in a delivery device as such. This invention also contemplates embodiments wherein the compositions are provided in a kit that stores one or more components separately from the remaining components. For example, in certain embodiments, the invention provides for a kit that separately stores botulinum toxin and the positively charged carrier for combining at or prior to the time of application. The amount of positively charged carrier or the concentration ratio of these molecules to the botulinum toxin will depend on which carrier is chosen for use in the composition in question. The appropriate amount or ratio of carrier molecule in a given case can readily be determined, for example, by conducting one or more experiments such as those described below.

In general, the invention also contemplates a method for administering botulinum toxin (alternatively as botulinum toxin complexes or reduced botulinum toxin complexes) to a subject or patient in need thereof, in which an effective amount of botulinum toxin is administered in conjunction with a positively charged carrier, as described herein. By “in conjunction with” it is meant that the two components (botulinum toxin and positively charged carrier) are administered in a combination procedure, which may involve either combining them prior to administration to a subject, or separately administering them, but in a manner such that they act together to provide the requisite delivery of an effective amount of the therapeutic protein. For example, a composition containing the positively charged carrier may first be administered to the skin of the subject, followed by application a skin patch, syringe, or other device containing the botulinum toxin. The botulinum toxin may be stored in dry form in a syringe or other dispensing device and the positively charged carrier may be injected before application of the toxin so that the two act together, resulting in the desired tissue penetration enhancement. In that sense, thus, the two substances (positively charged carrier and botulinum toxin) act in combination or perhaps interact to form a composition or combination in situ. Accordingly, the invention also includes a kit with a device for dispensing botulinum toxin and a liquid, gel, or the like that contains the positively charged carrier, and that is suitable for injection to the skin or target tissue of a subject. Kits for administering the compositions of the inventions, either under direction of a health care professional or by the patient or subject, may also include a custom applicator suitable for that purpose.

The compositions of this invention are suitable for use in physiologic environments with pH ranging from about 4.5 to about 6.3, and may thus have such a pH. However, compositions having a pH ranging from about 4.5 to about 7.5 are also embraced by the invention as described herein. The compositions according to this invention may be stored either at room temperature or under refrigerated conditions.

In some embodiments, the patient to be treated can any age for which treatment is appropriate, such as 18-90, 18-85, 18-80, 18-75, and 19-70.

### EXAMPLES

### Example 1

An injectable botulinum toxin formulation (DAXI) was prepared with ingredients according to Table 2 below. All inactive ingredients are listed in the US FDA inactive ingredients database, except for the positively charged peptide excipient with the sequence: RKKRRQRRRG-(K)₁₅-GRKKRRQRRR (SEQ ID NO: 4). The botulinum toxin molecule is not covalently associated with R004. RT002 does not contain accessory proteins or animal-derived proteins such albumin.

TABLE 2
Composition of DAXI for Injection

| | Quality | | | |
|---|---|---|---|---|
| Ingredient | Standard | Function | | |
| Botulinum toxin | Internal | Active | 100 U (0.50 ng^{d}) | |
| Type A, 150 kD | | Ingredient | | |
| L-histidine | USP/Ph. Eur. | Buffer | 0.14 | mg |
| L-histidine | Ph. Eur. | Buffer | 0.65 | mg |
| monohydrochloride | | | | |
| Trehalose dihydrate | NF/Ph. Eur. | Stabilizer | 36 | mg |
| Polysorbate-20 | NF/Ph. Eur. | Stabilizer | 0.1 | mg |
| RPT004 | Internal | Stabilizer | 11.7 | μg^{b} |

| | | | |
|---|---|---|---|
| Water for Injection | USP | Processing Aid | N/A^{c} |

^{a} Theoretical amount of toxn required for 50 U/vial drug product;
^{b}Equivalent to 15 μg RTP004-Acetate;
^{c}Water added in manufacturing is removed during the lyophilization step,
^{d}Theoretical amount of toxin required for 100 U/vial drug product.

A dose escalation study using the DAXI formulation is described in the Example section of PCT Publication No. WO2018/213710 to Revance Therapeutics, Inc. This example and the entire specification of the '710 Publication is hereby incorporated by reference in its entirety.

### Example 2—Clinical Study for Treating ULS with DAXI

Described herein is a randomized, double-blind, placebo-controlled, parallel group, dose-ranging clinical study to evaluate the efficacy and safety of DaxibotulinumtoxinA Injectable (DAXI) for treating upper limb spasticity in adults after stroke or traumatic brain injury. Eighty-three subjects with upper limb spasticity were enrolled at multiple sites in the United States. The trial's first cohort of 22 subjects received a single dose of 250 units of DAXI injectable, the second cohort of 19 subjects received a single dose of 375 units, and the third cohort of 18 subjects received a single dose of 500 units. The study showed positive efficacy results and that DAXI was generally safe and well-tolerated.

In this phase 2 efficacy and safety study, subjects were randomly assigned to 1 of 4 treatment groups (3 study drug groups of ascending doses and a placebo group) and the treatments were administered in double-blind fashion. While upper limb spasticity and associated symptoms can be debilitating for some subjects, the condition is not life threatening and employing a placebo group is important for establishing that any treatment effects are not related to a “placebo effect”, which can confound interpretation of results. In addition, to ensure that there is minimal to no carryover effect from prior BONT treatment of the paretic limb, subjects previously treated with BONTA (i.e., >16 weeks prior to Screening) must have a baseline Modified Ashworth Score (“MAS”) score of at least 3 at the suprahypertonic muscle group, which is the muscle group for which the co-primary efficacy endpoint of change in MAS was assessed at Week 6.

The treatment arms were diluted in a fixed injection volume of 5.0 mL. This volume is to allow for the injection of a sufficient number of clinically relevant muscles implicated in upper limb muscle spasticity tone, spasticity, and both passive and active function. Localization guidance (e.g., E-stimulation, EMG or ultrasonography) was used to ensure the targeted muscles of the affected limb are properly injected and to increase the efficacy and safety of the treatments (Gracies, et al., Arch Phys Me Rehabil. 2009 January; 90 (1): 9-16). The clinical assessment of muscle tone was performed using the MAS, which is a validated and widely used measure to evaluate muscle tone associated with spasticity (Bohannon, Phys Ther. 1987 February; 67 (2): 206-7; and Gregson, et al., Arch Phys Med Rehabil. 1999 September; 80 (9): 1013-6). Improvement in elbow, wrist and finger flexor muscle tone has been reported with Onabot, Abobot and Incobot (Brashear, et al., Neurol. 1999; 53:1439-1446; Gracies, et al., Lancet Neurol. 2015 October; 14 (10): 992-1001; and Elovic, et al., Muscle Nerve. 2016 March; 53 (3): 415-21). The literature supports a reduction of at least one-point in the MAS is considered clinically relevant (Gracies, et al., Lancet Neurol. 2015 October; 14 (10): 992-1001; Childers, et al., Arch Phys Med Rehabil. 2004 July; 85 (7): 1063-9; and Kanovsky, et al., Clin Neuropharmacol. 2009 September-October; 32 (5): 259-65). Impaired passive and active function are common in upper limb spasticity. The Disability Assessment Scale (“DAS”) measures spasticity related changes in four domains of disability: hygiene, dressing, limb position (cosmesis), and pain and is a rated using a scale of 0-3. A reduction of the DAS score contributes to improving activities of daily living, and it is considered clinically relevant (Brashear, et al., Arch Phys Med Rehabil. 2002 October; 83 (10): 1349-54; and Kanovsky, et al., Clin Neuropharmacol. 2009 September-October; 32 (5): 259-65). The DAS is supported by good intra- and inter-rater reliability (Brashear, et al., Arch Phys Med Rehabil. 2002 October; 83 (10): 1349-54). The Tardieu Scale (TS) was developed as a specific measure of spasticity (Gracies, et al., Arch Phys Med Rehabil. 2000 December; 81 (12): 1547-55). This validated measure of spasticity is based upon muscle responses to stretch at three different velocities, and passive muscles stretch is assessed at two velocities: slow and fast (Gracies, et al., Arch Phys Me Rehabil. 2009 January; 90 (1): 9-16; and Gracies, et al., Arch Phys Med Rehabil. 2010 March; 91 (3): 421-8). The clinical rater determines the spasticity angle (X)—the difference between the angles of arrest at slow speed and of catch-and-release or clonus at fast speeds; the spasticity grade (Y) is an ordinal variable that grades the intensity (gain) of the muscle reaction to fast speed. The slow velocity is below the threshold of the stretch reflex and measures the passive ROM, while the fast velocity reveals the stretch reflex in the presence of spasticity. The TS was recently used to assess the effectiveness of AboBoNTA in adults with upper limb spasticity (Gracies, et al., Lancet Neurol. 2015 October; 14 (10): 992-1001). The Fugl-Meyer Upper Extremity (FMUE) Scale is a widely used and highly recommended stroke-specific, performance-based measure of the active component of upper limb function. It is designed to assess reflex activity, movement control and muscle strength in the upper extremity of subjects with post-stroke hemiplegia. It has been extensively used as an outcome measure in rehabilitation trials and to record post-stroke recovery, particularly in the USA (Singer, Journal of Physiotherapy 63 (2017) 53). The duration of effect criteria utilized in this study are based upon the pivotal trial that supports the adult upper limb spasticity indication for abobotulinimtoxin A (Gracies, et al., Lancet Neurol. 2015 October; 14 (10): 992-1001; Isner-Horobeti, et al., Annals of Physical and Rehabilitation Medicine. 2017, 60:26-27; and Dysport {abobotulinumtoxinA} Package Insert, 2016).

Endpoints. The co-primary efficacy endpoints of the Phase 2 study was an improvement (e.g., mean change) from baseline in muscle tone measured using the Modified Ashworth Score (MAS) and the physician global impression of change (PGIC) in the suprahypertonic muscle group (SMG) of the elbow, wrist or finger flexors at week 6. MAS measures resistance of muscles during passive soft-tissue stretching, and is considered the gold standard for assessing spasticity. The MAS is performed in the supine position (this will garner the most accurate and the lowest scores as any tension anywhere in the body will increase spasticity). Because spasticity is “velocity dependent” (the faster the limb is moved, the more spasticity is encountered), the MAS is performed while moving the limb at the “speed of gravity”; this is defined as the same speed at which a non-spastic limb would naturally drop (fairly fast). The test is performed a maximum of three times for each joint; if more than three times, the short-term effect of a stretch can influence the score. The MAS is performed prior to goniometric testing; goniometric testing provides a stretch, and the short-term effect of a stretch can influence the score. The positions used for the MAS assessment are as follows.
a. Shoulder extensors. Start position: elbow as straight as possible, forearm neutral. Movement: flex the subject's shoulder from maximum possible extension to maximum possible flexion.
b. Shoulder internal rotators. Start position: shoulder adducted, elbow flexed to 90°, forearm neutral. Movement: externally rotate the subject's shoulder from neutral to maximum external rotation.
c. Shoulder adductors. Start position: elbow as straight as possible, forearm supinated. Movement: abduct the subject's arm from maximum possible adduction to maximum possible abduction.
d. Elbow. Start position: elbow fully flexed. Movement: extend the subject's elbow from maximum possible flexion to maximum possible extension.
e. Forearm. Start position: shoulder adducted, elbow flexed to 90° with palm facing down. Movement: supinate the subject's forearm by turning the palm up.
f. Wrist. Start position: elbow as straight as possible, forearm pronated. Movement: extend the subject's wrist from maximum possible flexion to maximum possible extension.
g. Fingers. Start position: elbow as straight as possible, forearm neutral. All fingers are done at once. Movement: extend the subject's fingers from maximum possible flexion to maximum possible extension.
h. Thumb (metacarpophalangeal joint). Start position: elbow as straight as possible, forearm neutral, wrist neutral. Movement: extend the thumb from maximum possible flexion to maximum possible extension.

Table 1 shows the scoring that MAS used. PGIC measures the clinician's overall impression of the subject's response or symptom change to study treatment since the start of the study medication. Table 2 shows the PGIC used.

TABLE 1
MAS

| Score | Modified Ashworth Score |
|---|---|
| 0 | Normal tone, no increase in tone |
| 1 | Slight increase in muscle tone, manifested by a catch and release |
| | or minimal resistance at the end of the range of motion (ROM) |
| | when the affected part(s) is moved in flexion and extension |
| 1+ | Slight increase in muscle tone, manifested by a catch, followed by |
| | minimal resistance throughout the remainder (less than half) of the |
| | ROM |
| 2 | More marked increase in muscle tone through most of the ROM, |
| | but affected part(s) easily moved |
| 3 | Considerable increase in muscle tone, passive movement difficult |
| 4 | Affected part(s) rigid in flexion or extension |

Subjects were eligible for study inclusion if they had a moderate to severe upper limb spasticity (ULS) with a MAS score ≥2 at the elbow, wrist, and finger flexors with the exception that the MAS is ≥3 at the SMG for subjects with prior BONTA in upper limb and spasticity angle ≥10 degrees in SMG.

TABLE 2
Physician Global Impression of Change
Physician Global Impression of Change (PGIC)
Please choose the response that best describes the overall change in your
subject's upper limb spasticity since the start of the study medication

| | |
|---|---|
| −4 | Markedly worse |
| −3 | Very much worse |
| −2 | Moderately worse |
| −1 | A little worse |
| 0 | About the same |
| 1 | A little improved |
| 2 | Moderately improved |
| 3 | Very much improved |
| 4 | Markedly improved |

The secondary efficacy endpoints of the Phase 2 study was (1) determining the proportion of subjects that showed an improvement by a full point on the MAS in the SMG at weeks 6 and 12; (2) determining the proportion of subjects that showed an improvement score of 1 full point or greater on the PGIC over time at weeks 6 and 12; (3) a change in functional impairment as measured by the disability assessment score (DAS) for the principal treatment target (PTT) at weeks 6 and 12; (4) determining the duration of effect; and (5) safety. The duration of effect of DAXI is defined as time from injection (in weeks) until there is a loss of muscle tone improvement in the SMG (i.e., a reduction from baseline in MAS score of >1 point) and the PGIC score is ≤0.

Study design. This randomized, double-blind, placebo-controlled trial evaluated the dose-related efficacy and safety of 3 doses of DAXI for injection for the treatment of upper limb spasticity (ULS) in adult subjects.

Adult subjects with upper limb spasticity after stroke or traumatic brain injury were screened. Eligible subjects with ULS were characterized by a primary aggregate posture (AP) of a flexed elbow+flexed wrist+clenched first (flexed fingers); each muscle group with a MAS score ≥2 (with the exception that the MAS score is ≥3 at the suprahypertonic muscle group [i.e., the muscle group with the highest baseline MAS score] for subjects with previous injections of BONTA in the paretic limb) and at least one other clinical pattern (adducted and internally rotated shoulder, pronated forearm or thumb-in-palm deformity) with a MAS score ≥1. Subjects also have moderate to severe functional impairment (DAS) score ≥2 in at least 1 of 4 functional domains (hygiene, dressing, limb position, or pain). At baseline, the functional domain for each subject's principal treatment target was selected. Subjects may be toxin-naïve or previously treated. Eligible subjects were randomized (1:1:1:1) to one of four treatment groups: Group 1: DAXI 250 U (N=22); Group 2: DAXI 375 U (N=19); Group 3: DAXI 500 U (N=18); Group 4: Placebo (N=24). Subjects were also be stratified by prior BoNT treatment experience and treatment center. The subject, Sponsor, and study site research personnel were blinded to the identity of the subject's assigned treatment.

On Day 1, baseline measurements were recorded, before subjects received intramuscular injections of DAXI or placebo according to their assigned treatment group. The baseline measurements are summarized below:
a. Vital signs (blood pressure [BP], pulse, temperature) and weight
b Physical and neurological examinations
c. Concomitant medications/therapies (including OT/PT), medical history information
d. Urine pregnancy test (UPT) for WOCBP only. If UPT is positive, obtain SPT to confirm
e. Columbia-Suicide Severity Rating Scale (C-SSRS)—Baseline/Screening version
f. Modified Ashworth Scale for rating muscle tone in the AP (elbow, wrist, and finger flexors) and determine the SMG and the muscles of the other clinical pattern
g. Disability Assessment Score (DAS) for measuring passive limb function.
h. Spasticity per the Tardieu Scale (TS) for rating spasticity in the SMG and in the AP and other clinical patterns.
i. Active Range of Motion (AROM) via goniometer against the SMG.
j. Active function via the Fugl-Meyer Extremity (FMUE) Assessment for rating active upper limb function.
k. EuroQOL Five Dimension Scale (EQ-5D-5L).
l. Short Form Health Survey (SF-36)

Electromyography, ultrasonography and/or nerve stimulation technique was used to identify the target muscles of ULS for injection. A total of 5.0 mL of reconstituted study drug for injection was injected into the identified target muscles. Study drug solution was divided and injected in the following manner: 3.5 mL injected into the identified muscles of the primary aggregate posture: the elbow flexors (1.5 mL), wrist flexors (1.0 mL), and finger flexors (1.0 mL) and the remaining 1.5 mL was injected into at least one of the identified hypertonic muscle group (MAS≥1) of the other clinical patterns: the adducted and internally rotated shoulder, pronated forearm, and/or palm-in-hand deformity. Table 5 shows injection parameters including a list of specific injectable muscles.

After the baseline visit, follow up assessments occurred at weeks 2, 4, 6, 8 and 12 (for the subjects) and weeks 16, 20, 24, 28, 32, and/or 36 (for the subset of subjects who have maintained the treatment benefit or have not reached the endpoint for duration of effect at week 12). Because some subjects may have entered the study after COVID-19 some subjects exited the study at week 12.

Eligibility Criteria. Inclusion Criteria:
1. Adults, 18 to 75 years of age, inclusive.
2. Written informed consent including authorization to release health information.
3. Focal upper limb spasticity (ULS) after a stroke (as defined by WHO criteria) or traumatic brain injury (TBI), last stroke or TBI>24 weeks prior to screening.
4. ULS with the primary aggregate posture (AP): flexed elbow+flexed wrist+clenched first (flexed fingers) and ≥1 other clinical pattern(s): adducted and internally rotated shoulder, pronated forearm, and/or thumb-in-palm deformity.
5. Moderate to severe ULS with a MAS score ≥2 at the elbow, wrist, and finger flexors; with the exception that the MAS score is ≥3 at the suprahypertonic muscle group (SMG) for subjects with previous injections of BONTA in the paretic limb.
6. Moderate to severe functional disability (DAS score ≥2) on the principal target of treatment (1 of 4 functional domains: hygiene, dressing, malposition of the arm/wrist/fingers, and pain).
7. Has sufficient cognitive and communication ability to be able to give informed consent including authorization to release health information.
   Eligibility Criteria. Exclusion Criteria:
1. Upper limb spasticity attributable to an etiology other than stroke or TBI.
2. Bilateral upper limb paresis or quadriplegia.
3. Initiated in physiotherapy of the upper extremities <30 days prior to screening or planned to start physiotherapy of the upper extremities during the course of the study.
4. Previous treatment with any BONT product for any condition <16 weeks prior to screening.
5 Botulinum neurotoxin treatment-experienced subjects who have historically required <200 U of Botox/Xeomin or its equivalent to effectively treat the upper limb spasticity.
6. Botulinum neurotoxin treatment-experienced subjects who had suboptimal or no treatment response to the most recent BoNTA injection for spasticity; or history of primary or secondary non-response to BoNTA injections, known to have neutralizing antibodies to BoNTA; or have a history of botulinum toxin type B (rimabotulinumtoxinB [Myobloc/Neurobloc]) injection for spasticity due to non-response or suboptimal response to BONTA.
7. Change in oral medications for spasticity including dosage and dosing frequency <30 days prior to screening.
8. Previous or planned treatment of the spastic upper limb with phenol, alcohol injection or surgery.
9. Profound muscular atrophy or fixed contracture (spasticity angle, per the Tardieu Scale, <10 degrees in the most hypertonic muscle group {elbow, wrist or finger flexors}) of the spastic limb leading to marked limitation on passive range of motion or any other known conditions of the upper limb that could confound muscle tone or functional assessment.
10. Current or prior treatment with intrathecal baclofen pump.
11. Any neuromuscular neurological conditions that may place the subject at increased risk of morbidity with exposure to BoNT, including peripheral motor neuropathic diseases (e.g., amyotrophic lateral sclerosis and motor neuropathy, and neuromuscular junctional disorders such as Lambert-Eaton syndrome and myasthenia gravis).
12. Use of aminoglycoside antibiotics, polymyxins, lincosamides (e.g., clindamycin), or other agents that might interfere with neuromuscular transmission (e.g., curare-like drugs, quinidine, magnesium sulfate, anticholinesterases, succinylcholine chloride)<14 days prior to Screening.
13. Women of child bearing potential (WOCBP), who have a positive pregnancy test at Screening or Baseline, do not agree to use an effective method of birth control, or plan to become pregnant during the course of the study.
14. Female, who is pregnant or nursing (lactating).
15. Participants in an investigational drug or device study <30 days prior to Screening.
16. Active skin infections at the injection sites which would put the subject at increased risk of morbidity with BoNT injections.
17. Planned treatment of other conditions (e.g. wrinkles, lower limb spasticity, sialorrhea, over-active bladder, neurogenic detrusor overactivity, cervical dystonia, chronic migraine, and hyperhydrosis) with BoNT during the course of the study.
18. Known to have sensitivity to any component of study medication (Clostridium botulinum toxin type A and/or excipients of DAXI for injection).
19. History of bleeding disorders that are not well-controlled, or on anticoagulation treatment with international normalized ratio (INR)>3.5 at Screening.
20. History of respiratory symptoms from congestive heart failure, chronic obstructive pulmonary disease, restrictive lung disease, or any unstable pulmonary disease <30 days prior to Screening.
21. History of Torsade de Pointe, Long QT Syndrome, secondary degree AV block Mobitz type II, or complete heart block.
22. History of severe sialorrhea (Drooling Severity and Frequency Scale score ≥8), increasing the risk for complications associated with aspiration (e.g. pneumonia, respiratory depression/failure).
23 Body weight of <54.4 kg.
24 Any acute illnesses or medical conditions including cognitive impairment (e.g., dementia), significant psychiatric illnesses or symptoms (e.g., suicidal ideation, psychosis) that are not stable and may put the subject at increased risk of morbidity.

Table 3 shows the subjects included in the study, Table 4 shows the subject demographics, and Table 5 shows the ULS baseline characteristics.

TABLE 3
Subject disposition.

| | Placebo | | DAXI 250 U | DAXI 375 U | DAXI 500 U | All Subjects | |
|---|---|---|---|---|---|---|---|
| N (%) | (N = 24) | | (N = 22) | (N = 19) | (N = 18) | (N = 83) | |
| Randomized Subjects | 24 | | 22 | 19 | 18 | 83 | |
| Study Completers | 10 | (41.7) | 9 (40.9) | 6 (31.6) | 9 (50.0) | 34 | (41.0) |
| Reached Week 36/Week 12 | | | | | | | |
| Early Completers | 8 | (33.3) | 6 (27.3) | 6 (31.6) | 6 (33.3) | 26 | (31.3) |
| Reached Target MAS Score | | | | | | | |
| Early Discontinuation | 6 | (25.0) | 7 (31.8) | 7 (36.8) | 3 (16.7) | 23 | (27.7) |
| Withdrawal of Consent | 1 | (4.2) | 4 (18.2) | 1 (5.3) | 0 | 6 | (7.2) |
| Lost to Follow-up | 0 | | 1 (4.5) | 0 | 0 | 1 | (1.2) |
| Physician Decision | 1 | (4.2) | 0 | 0 | 0 | 1 | (1.2) |
| Sponsor Request | 1 | (4.2) | 2 (9.1) | 5 (26.3) | 2 (11.1) | 10 | (12.0) |
| (Amendment 3) | | | | | | | |
| Lack of Efficacy | 1 | (4.2) | 0 | 1 (5.3) | 1 (5.6) | 3 | (3.6) |
| Protocol Violation | 1 | (4.2) | 0 | 0 | 0 | 1 | (1.2) |
| Other | 1 | (4.2) | 0 | 0 | 0 | 1 | (1.2) |

TABLE 4
Subject Demographics

| | Placebo | DAXI 250 U | DAXI 375 U | DAXI 500 U | All Subjects |
|---|---|---|---|---|---|
| | (N = 24) | (N = 22) | (N = 19) | (N = 18) | (N = 83) |
| Female, n (%) | 12 (50.0) | 7 (31.8) | 10 (52.6) | 5 (27.8) | 34 (41.0) |
| Age (years) | 54.1 (9.52) | 51.4 (8.08) | 51.9 (10.71) | 55.9 (7.62) | 53.3 (9.08) |
| mean (SD) range | 22, 66 | 32, 65 | 19, 63 | 42, 67 | 19, 67 |
| Race, n (%) | | | | | |
| White | 14 (58.3) | 17 (77.3) | 11 (57.9) | 14 (77.8) | 56 (67.5) |
| Black/African American | 10 (41.7) | 4 (18.2) | 6 (31.6) | 3 (16.7) | 23 (27.7) |
| Asian | 0 | 1 (4.5) | 1 (5.3) | 0 | 2 (2.4) |
| Other* | 0 | 0 | 1 (5.3) | 1 (5.6) | 2 (2.4) |

*includes “Other”, “Not Reported”

TABLE 5
ULS baseline characteristics.

| | Placebo | DAXI 250 U | DAXI 375 U | DAXI 500 U | All Subjects |
|---|---|---|---|---|---|
| | (N = 24) | (N = 22) | (N = 19) | (N = 18) | (N = 83) |
| ULS Diagnosis due to, n (%) | | | | | |
| Stroke | 21 (87.5) | 22 (100) | 18 (94.7) | 18 (100) | 79 (95.2) |
| Traumatic Brain Injury | 3 (12.5) | 0 | 1 (5.3) | 0 | 4 (4.8) |
| (TBI) | | | | | |
| Duration of ULS (years) | 7.7 (9.37) | 10.8 (9.91) | 6.6 (5.29) | 8.0 (8.70) | 8.3 (8.61) |
| Mean (SD) Range | 0.0, 40.5 | 1.4, 38.2 | 0.5, 21.6 | 0.0, 38.3 | 0.0, 40.5 |
| Prior Toxin Treatment | | | | | |
| for ULS, n (%) | | | | | |
| Yes | 15 (62.5) | 14 (63.6) | 15 (78.9) | 14 (77.8) | 58 (69.9) |
| No | 9 (37.5) | 8 (36.4) | 4 (21.1) | 4 (22.2) | 25 (30.1) |
| SMG MAS Score Mean (SD) | 4.0 (0.29) | 4.0 (0.38) | 4.0 (0.33) | 3.9 (0.32) | 4.0 (0.33) |

Product, dose, and route of administration. DaxibotulinumtoxinA for injection (lyophilized powder for reconstitution with 0.9% non-preserved, normal saline solution): 250 U, 375 U, and 500 U; intramuscular injections.

DaxibotulinumtoxinA for injection (250 U, 375 U or 500 U) or placebo was administered by IM injections in the affected muscles of the upper limb to increase the range of motion of the affected limb, improve hygiene and other activities of daily living.

DaxibotulinumtoxinA for injection is an acetylcholine release inhibitor and neuromuscular blocking agent. It is composed of purified 150 kDa BoNTA formulated with the proprietary stabilizing excipient peptide shown in SEQ ID NO: 4. It is further formulated in a lyophilized product to be reconstituted with sterile, non-preserved 0.9% sodium chloride solution for IM injection. Reconstituted product is a clear, colorless, solution free of particulates. After reconstitution, the product can be stored at room temperature and used within 2 hours of preparation. The placebo is a lyophilized product containing inactive ingredients that can be reconstituted with sterile, non-preserved 0.9% sodium chloride solution for IM injection.

Formulation and appearance. DaxibotulinumtoxinA for injection is composed of 150 kDa of a purified BoNTA and excipients including the peptide according to SEQ ID NO: 4. It is provided as a lyophilized powder in a sterile 2 mL type 1 borosilicate glass, single-use vial. The placebo containing inactive ingredients without the botulinum neurotoxin is also provided as a lyophilized powder in a sterile 2 mL type 1 borosilicate glass, single-use vial. Sterile, preservative-free, 0.9% sodium chloride solution is provided for reconstitution of the DAXI formulation.

Storage and handling. DaxibotulinumtoxinA for injection should be stored at 2 to 8° C. (36 to 46° F.) until use and should not be frozen. Vials should be stored upright and protected from light. Storage should be in a secure location, such as a locked refrigerator.

Reference therapy, dose and route of administration. Placebo for injection (lyophilized powder for reconstitution with 0.9% non-preserved, normal saline solution): 0 U; intramuscular injections.

Injection parameters. The muscles that can be selected for injection are listed in the Table 5. The muscles for injection were identified using EMG, ultrasonography, or nerve stimulation techniques. The injection volume by muscle group corresponds to a specific dose, respectively, for DAXI for injection 250 U, 375 U, and 500 U. Table 6 shows the recommended number of injection sites per muscle.

The 5.0 mL solution of reconstituted study drug was injected as follows: 3.5 mL for the muscles of the primary aggregate posture distributed as follow: 1.5 mL for the flexed elbow, 1.0 mL for the flexed wrist, 1.0 mL for the clenched first (flexed fingers), and 1.5 mL for muscles of other clinical patterns. No more than 1.0 mL was be injected into a single muscle group of other clinical patterns.

The fixed injection volume (3.5 mL or 70% of available volume): common pattern of the aggregate posture with MAS≥2 (flexed elbow+flexed wrist+clenched fist).

The variable injection volume (1.5 mL or 30% of available volume): other clinical pattern with MAS≥1 (adducted/internally rotated shoulder, pronated forearm, thumb-in-palm).

TABLE 6
Fixed and variable injection volume.

| | Injection | | | | | | |
|---|---|---|---|---|---|---|---|
| | Volume | | Placebo | DAXI 250 U | DAXI 375 U | | DAXI 500 U |
| Pre-defined Injection Volume | | | | | | | |
| Mandatory fixed injection volume: | 3.5 | mL | 0 unit | 175 units | 263 | units | 350 units |
| 70% | | | | | | | |
| Mandatory variable injection volume: | 1.5 | mL | 0 unit | 75 units | 112 | units | 150 units |
| 30% | | | | | | | |
| Total mandatory injection volume | 5.0 | mL | 5.0 mL | 5.0 mL | 5.0 mL | | 5.0 mL |
| Final Dilution | | | 0 U/0.1 mL | 5 U/0.1 mL | 7.5 U/0.1 mL | | 10 U/0.I mL |
| Primary Aggregate Posture (AP): | | | | | | | |
| Injectable Muscles | | | | | | | |
| Flexed elbow | 1.5 | mL* | 0 unit | 75 units | 112 | units | 150 units |
| Biceps; Brachioradialis; Brachialis | | | | | | | |
| Flexed wrist | 1.0 | mL* | 0 unit | 50 units | 75 | units | 100 units |
| Flexor carpi radialis; Flexor carpi | | | | | | | |
| ulnaris | | | | | | | |
| Clenched fist | 1.0 | mL* | 0 unit | 50 units | 75 | units | 100 units |
| Flexor digitorum superficialis; Flexor | | | | | | | |
| digitorum profundus | | | | | | | |
| Other Clinical Patterns: Injectable | 1.5 | mL* | 0 unit | 75 units | 112 | units | 150 units |
| Muscles | | | | | | | |

| | | | | |
|---|---|---|---|---|
| Adducted & internally rotated | 0 unit | Up to 50 | Up to 75 | Up to 100 |
| shoulder | | units | units | units |
| Pectoralis complex; Latissimus dorsi | | | | |
| Pronated forearm | 0 unit | Up to 50 | Up to 75 | Upto 100 |
| | | units | units | units |
| Pronator quadratus; pronator teres | | | | |
| Thumb-in-palm | 0 unit | Up to 25 | Up to 37 | Up to 50 |
| | | units | units | units |
| Flexor pollicis longus; Adductor | | | | |
| pollicis; Flexor pollicis | | | | |
| brevis/opponens; first interosseous | | | | |

*Fixed injection volume to be divided among the injectable muscles of the flexed elbow, flexed wrist, and clenched fist.
†Variable injection volume to be divided among the injectable muscles of ≥1 other clinical pattern.

Selection of suprahypertonic muscle group (SMG). Each subject had one muscle group (elbow, wrist, or fingers) designated as primary, known as the Suprahypertonic Muscle Group (SMG). The SMG was the muscle group with the highest MAS score at baseline, excluding those with a Tardieu Scale Spasticity Angle <10 (which indicates severely restricted range of motion and potentially a fixed contracture). Screening MAS and Tardieu scores were reviewed for eligibility. Subjects were screen-failed if the muscle group with the highest MAS score also had a Tardieu <10. After database lock, 7 subjects were found to have an incorrectly identified SMG as defined by the protocol. Two had an SMG that did not have the highest MAS score at baseline and five had an SMG with a baseline Tardieu score <10. SMG assignment was corrected with statistical programming.

TABLE 7
Number of injection sites per muscle.

| | | Recommend number of |
|---|---|---|
| Flexor | Muscles | injection sites |
| Elbow | Biceps brachii | 1-4 |
| | Brachials | 1-2 |
| | Brachioradialis | 1-3 |
| Wrist | Flexor carpi radialis | 1-2 |
| | Flexor carpi ulnaris | 1-2 |
| Finger | Flexor digitorum profundus | 2 |
| | Flexor digitorum superficialis | 2 |
| Forearm | Pronator teres | 1-2 |
| | Pronator quadratus | 1 |
| Thumb | Flexor pollicis longus | 1 |
| | Adductor pollicis | 1 |
| | Flexor pollicis brevis | 1 |

### Results.

Efficacy summary. DAXI administration improved the co-primary endpoints.

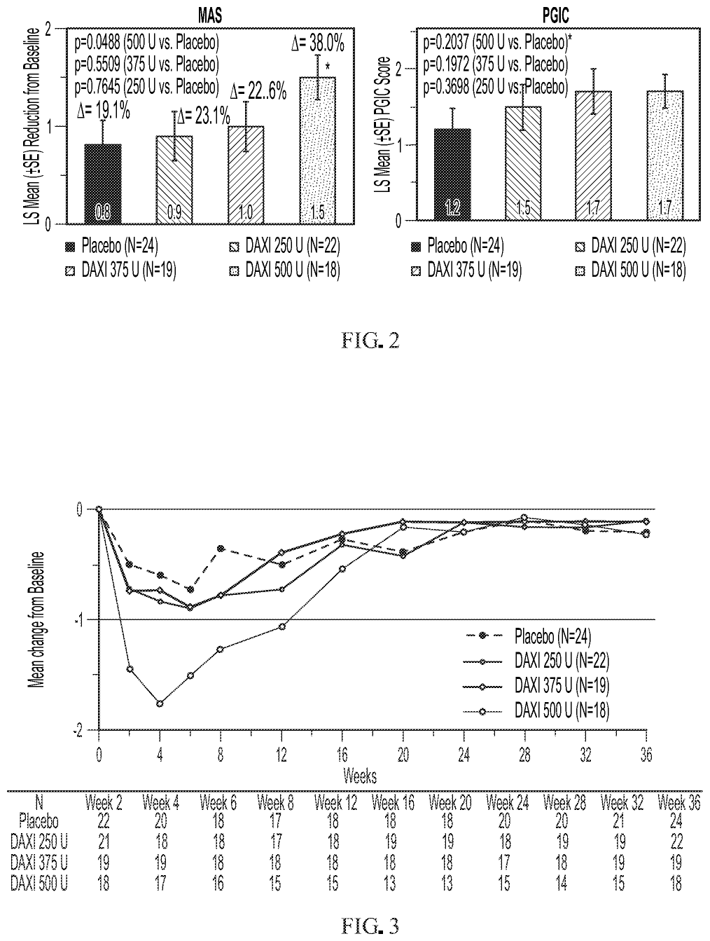

MAS change from baseline. A statistically significant and clinically meaningful difference in MAS improvement from baseline for the SMG at week 6 was observed for the DAXI 500 U group (1.5 points or 38%) compared with placebo (0.8 points or 19.1%, p=0.0488) (FIG. 2). The improvement from baseline for DAXI 250U (0.9 or 23.1%) and 375U (1.0 or 22.6%) vs. placebo was not significant. Analysis of individual muscle groups, regardless of SMG designation, indicated a statistically significant improvement in the wrist and finger in the DAXI 500U group vs. placebo at week 6. The elbow muscle group did not show an improvement in the MAS score v. placebo over the treatment period.

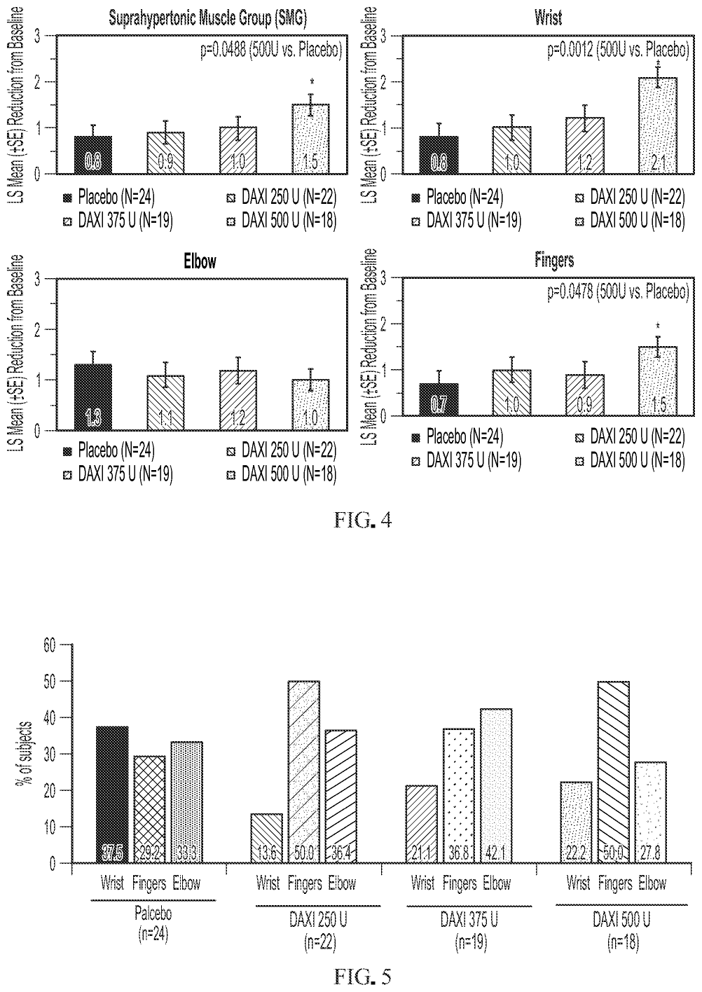

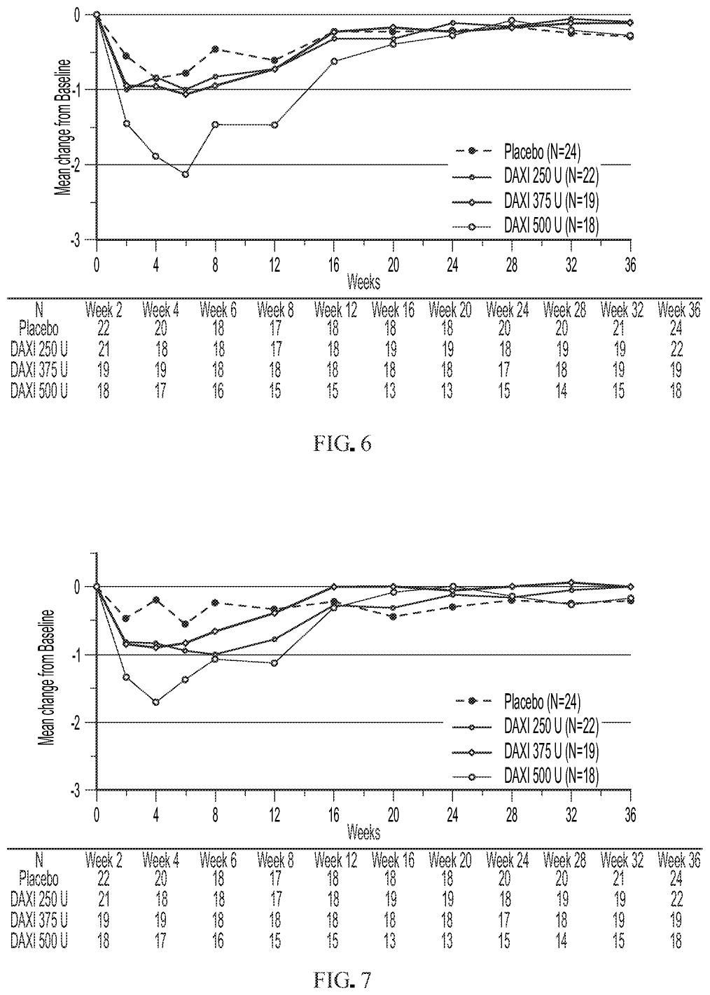

DAXI 500U showed the greatest mean change from baseline on the SMG MAS score over time compared to placebo (FIG. 3). DAXI 500U also showed the greatest mean change in MAS from baseline at week 6 for SMG, wrist, and fingers compared to placebo (FIG. 4). No change was observed in MAS from baseline at week 6 for elbow, suggesting that a higher dose may be needed. FIG. 5 shows the SMG selection for each DAXI dose. The mean change from baseline on MAS over time for wrist, fingers, and elbow are shown FIGS. 6-8, respectively. While DAXI 500U showed a change from baseline on MAS over time for wrist compared to placebo, and DAXI doses 250U, 375U and 500U each showed an improvement from baseline on MAS over time for fingers, no change was observed for any of the doses tested for elbow, suggesting a higher does may be needed.

Physician global impression of change. A numerical trend towards higher ratings of improvement on the PGIC at week 6 were observed for DAXI 250U, 375U and 500U (means of 1.5, 1.7 and 1.7, respectively) compared with placebo (1.2, p>0.05 for the DAXI groups vs. placebo).

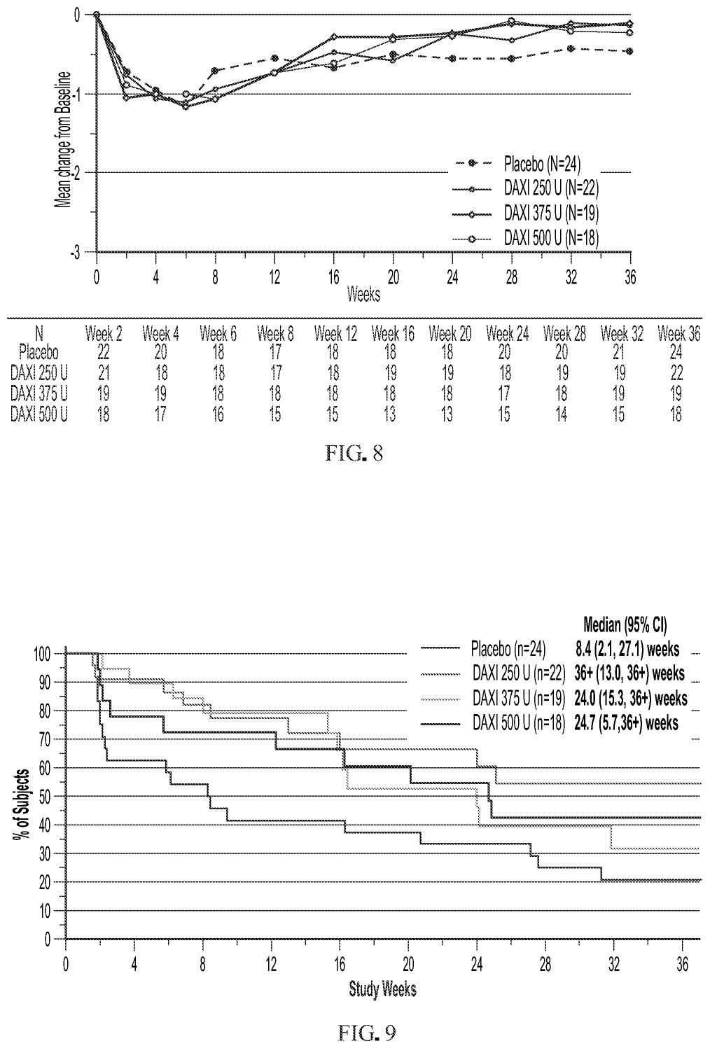

Duration of effect. Median duration of effect, defined as time to loss of improvement in SMG (MAS change from baseline of <1) and PGIC≤0 or requesting retreatment, was 36+, 24.0 and 24.7 weeks for DAXI 250U, 375U and 500U, respectively. Currently approved BoNT-A products uniformly report a median duration of effect of 12-14 weeks between treatment intervals for the treatment of adult ULS. FIG. 9 shows the time to loss of improvement in suprahypertonic muscle group (SMG) (modified Ashworth score (MAS) change from baseline <1 point) and physician global impression of change (PGIC)≤ or requesting retreatment. The results show that the median effect for DAXI 250 U, 375 U, and 500 U were long lasting (more than 36 weeks, 24 weeks, and 24.7 weeks, respectively) compared to placebo.

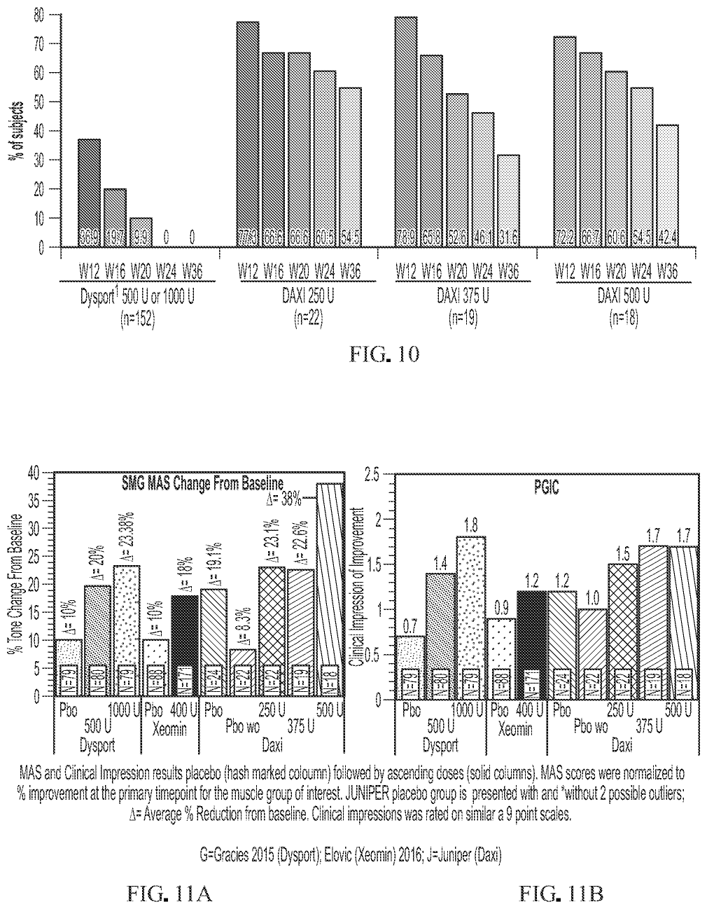

The duration of effect of DAXI at 250U, 375U, and 500U was compared to published results using Dysport (Esquinazi A., Frontiers in Neurology, 2020, data from OLS cycle 1). The results show that the proportion of subjects requiring retreatment of DAXI at weeks 12, 16, 20, 24, and 36 weeks was lower at each dose of DAXI compared to Dysport®, suggesting that DAXI has a longer duration of effect than Dysport (see, FIG. 10).

Safety summary. DAXI at 250U, 375U and 500U doses was generally safe and well tolerated through week 36 with no trend toward increased incidences of adverse events with increased doses. Treatment-related adverse events occurred in 20.8%, 18.2%, 15.8% and 11.1% of subjects in the placebo, 250U, 375U and 500U dose groups, respectively. The majority of treatment related adverse effects's were mild or moderate in severity. No new or unexpected treatment emergent adverse events were observed. Muscle weakness occurred in 2 subjects (9.1%) in the 275 U group, with a single report of muscle weakness in the placebo (4.2%) and 500 U (5.6%) groups. Other adverse events occurred in a single subject in any treatment group. The incidence of treatment-related adverse effects was similar or lower compared with prior BONTA studies in adult upper limb spasticity. No serious adverse events related to study treatment were reported. Table 8 provides a summary of the adverse events of the study and Table 9 provides further details on the treatment related adverse events.

TABLE 8
Adverse Events

| | Placebo | DAXI 250 U | DAXI 375 U | DAXI 500 U | All Subjects |
|---|---|---|---|---|---|
| | (N = 24) | (N = 22) | (N = 19) | (N = 18) | (N = 83) |
| Adverse Event Category | n (%) E* | n (%) E | n (%) E | n (%) E | n (%) E |
| Any TEAEs | 13 (54.2) 26 | 12 (54.5) 24 | 12 (63.2) 19 | 7 (38.9) 16 | 44 (53.0) 85 |
| Treatment-Related TEAEs | 5 (20.8) 8 | 4 (18.2) 4 | 3 (15.8) 4 | 2 (11.1) 3 | 14 (16.9) 19 |
| Serious TEAEs | 2 (8.3) 2 | 2 (9.1) 2 | 0 | 1 (5.6) 1 | 5 (6.0) 5 |
| Treatment-Related Serious | 0 | 0 | 0 | 0 | 0 |
| TEAEs | | | | | |
| Any Potential Distant Spread | 1 (4.2) 1 | 3 (13.6) 3 | 2 (10.5) 2 | 2 (11.1) 2 | 8 (9.6) 8 |
| of Toxin TEAEs (based on | | | | | |
| SMQ) | | | | | |
| Any Potential TEAEs related | 0 | 0 | 0 | 0 | 0 |
| to Anaphylaxis (based on | | | | | |
| SMQ) | | | | | |
| Death | 0 | 0 | 0 | 0 | 0 |
| TEAEs Leading to Study | 0 | 0 | 0 | 0 | 0 |
| Discontinuation | | | | | |

*incidence (rate) and number of events

TABLE 9
Treatment-related Adverse Events

| | Placebo | DAXI 250 U | DAXI 375 U | 531 DAXI 500 U | All Subjects |
|---|---|---|---|---|---|
| | (N = 24) | (N = 22) | (N = 19) | (N = 18) | (N = 83) |
| Adverse Event Category | n (%) E* | n (%) E | n (%) E | n (%) E | n (%) E |
| Subjects with any | 5 (20.8) 8 | 4 (18.2) 4 | 3 (15.8) 4 | 2 (11.1) 3 | 14 (16.9) 19 |
| Treatment- Related | | | | | |
| Treatment-Emergent | | | | | |
| Adverse Events | | | | | |
| Injection site bruising | 1 (4.2) 1 | 1 (4.5) 1 | 1 (5.3) 1 | 0 | 3 (3.6) 3 |
| Injection site erythema | 0 | 0 | 1 (5.3) 1 | 0 | 1 (1.2) 1 |
| Injection site haematoma | 0 | 0 | 0 | 1 (5.6) 1 | 1 (1.2) 1 |
| Injection site induration | 0 | 0 | 0 | 1 (5.6) 1 | 1 (1.2) 1 |
| Injection site swelling | 0 | 0 | 1 (5.3) 1 | 0 | 1 (1.2) 1 |
| Muscular weakness | 0 | 2 (9.1) 2 | 0 | 1 (5.6) 1 | 3 (3.6) 3 |
| Ecchymosis | 0 | 0 | 1 (5.3) 1 | 0 | 1 (1.2) 1 |

*incidence (rate) and number of events

Results after Removal Placebo Outliers:

A very strong placebo effect was observed from 2 of the 24 in the placebo group. Specifically, as shown in the red circle in the table on the right, a decrease in the MAS from baseline of 3 and 4 points was observed. Comparing such results to that of a Dysport® Phase 2 Study for treating ULS show that such a strong placebo effect is atypical.

TABLE 10a
Dysport Summary
TABLE 3. Summary of Best Change at Week 4 From Baseline
in the MAS and Odds Ratio for Improvement in MAS for Each
Dysport Group Compared With Placebo

| | | Dysport | | |
|---|---|---|---|---|
| Change in MAS | Placebo | 500 U | 1000 U | 1500 U |
| From Baseline | [symbol] | [symbol] | [symbol] | [symbol] |
| Decrease of 4 | 0 | 0 | [symbol] | [symbol] |
| Decrease of 3 | 0 | [symbol] | [symbol] | [symbol] |
| Decroose of 2 | [symbol] | [symbol] | [symbol] | [symbol] |
| Decrease of 1 | [symbol] | [symbol] | [symbol] | [symbol] |
| No change | [symbol] | [symbol] | [symbol] | [symbol] |
| Increase of 1 | 0 | 0 | [symbol] | 0 |
| Odds ratio | | [symbol] | [symbol] | [symbol] |
| 95% CI | | [symbol] | [symbol] | [symbol] |
| P | | 0.02 | 0.001 | 0.02 |

[symbol]

indicates data missing or illegible when filed

TABLE 10b
Daxi Study
Summary of Change from Baseline in the SMG MAS and %
Responders for Each Treatment Group at Week 6

| | | DAXI | | |
|---|---|---|---|---|
| Change | Placebo | 250 U | 375 U | 500 U |
| in MAS From Baseline | (N = 24) | (N = 22) | (N = 19) | (N = 18) |
| Decrease of 4 Decrease of 3 | 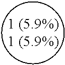 | 0 0 | 0 3 (5.9% | 1 (6.3%) 5 (31.3%) |
| Decrease of 2 | 0 | 3 (16.7%) | 3 (17.6%) | 0 |
| Decrease of 1 | 6 (35.3%) | 10 (55.6%) | 7 (41.2%) | 5 (31.3%) |
| Nu change | 9 (52.9%) | 5 (27.8%) | 6 (35.3%) | 5 (31.3%) |
| Responders N (%*) | 8 (47.1) | 13 (72.2) | 11 (64.7) | 11 (68.8) |
| (at least 1 point | | | | |
| improvement) | | | | |
| Totals | 17 | 18 | 17 | 16 |

[symbol]

[symbol]

indicates data missing or illegible when filed

Reanalysis of the results with and without the placebo outliers is shown below. FIGS. 11a and 11b show both the improvements of MAS and PGIC scores with the 2 placebo outliers (see column labeled “pbo”) and without the 2 placebo outliers (see column labeled “pbo wo”) at time point of week 4 or week 6 after treatment. FIGS. 11a and 11b show the similar data for Xeomin® and Dysport® by way of comparison. As can be seen, the placebo effect in both the Xeomin® and Dysport® trials is similar to the placebo effect of the present study without the placebo outliers (see colum “pbo wo”).

It is understood that the following examples and embodiments described herein are for illustrative purposes and that various modifications or changes in light thereof will be suggested to persons skilled in the art and are to be included within the spirit and purview of this application and scope of the appended claims.

All publications, patents, and published patent applications cited herein are hereby incorporated by reference in their entireties for all purposes.

1.-36. (canceled)

37. A method of treating upper limb spasticity in an individual, the method comprising:
administering by injection a treatment dose of the sterile injectable composition, wherein the composition comprises a botulinum toxin of serotype A having a molecular weight of 150 kDa into one or more muscles contributing to the upper limb spasticity in the individual in need of treatment to achieve the therapeutic effect;
wherein the composition is albumin-free and comprises a pharmaceutically acceptable diluent suitable for injection, a non-ionic surfactant, and the botulinum toxin;
wherein the total treatment dose of botulinum toxin administered to the individual is between 250 U to 1000 U;
wherein the composition comprises a positively charged carrier comprising a positively charged backbone having covalently attached thereto one or more positively charged efficiency groups;
wherein the duration of treatment effect is at least 16 weeks.

38. The method according to claim 37, wherein one or more types of upper limb spasticity are treated and wherein the one or more types of upper limb spasticity is selected from a clenched fist, flexed fingers, flexed elbow, flexed wrist, adducted and internally rotated shoulder, and pronated forearm.

39. The method according to claim 38, wherein the individual presents with the one or more types of upper limb spasticity selected from a group consisting of (i) flexed elbow, flexed wrist, and a clenched first or (ii) flexed elbow, flexed wrist, and flexed fingers.

40. The method according to claim 38, wherein the individual presents with upper limb spasticity comprising a flexed fingers or clenched fist, wherein the treatment dose for flexed fingers or clenched comprises 50 to 100 U, wherein the one or more muscles injected for treatment of flexed fingers or clenched first comprises flexor digitorum superficialis and flexor digitorum profundus.

41. The method according to claim 38, wherein the individual presents with upper limb spasticity comprising a flexed wrist, wherein the treatment dose for flexed wrist comprises 50 to 100 U, wherein the one or more muscles injected for treatment of flexed wrist comprises flexor carpi radialis and flexor carpi ulnaris.

42. The method according to claim 38, wherein the individual presents with upper limb spasticity comprising a flexed elbow, wherein the treatment dose for flexed elbow comprises 75 U to 150 U, wherein the one or more muscles injected for treatment of flexed elbow comprises biceps; brachioradialis; and brachialis.

43. The method according to claim 38, wherein the individual presents with upper limb spasticity comprising an adducted and internally rotated shoulder, wherein the treatment dose for adducted and internally rotated shoulder comprises 50 U to 100 U, wherein the one or more muscles injected for treatment of adducted and internally rotated shoulder comprises pectoralis complex and latissimus dorsi.

44. The method according to claim 38, wherein the individual presents with upper limb spasticity comprising a pronated forearm, wherein the treatment dose for pronated forearm comprises 50 U to 100 U, wherein the one or more muscles injected for treatment of pronated forearm comprises pronator quadratus and pronator teres.

45. The method according to claim 38, wherein the individual presents with upper limb spasticity comprising a thumb in palm, wherein the treatment dose for thumb-in-palm comprises 25 U to 50 U, wherein the one or more muscles injected for treatment of thumb-in-palm comprise flexor pollicis longus, adductor pollicis; flexor pollicis brevis/opponens, and first interosseous.

46. The method according to claim 38, wherein the risk of adverse events is less than 5%.

47. The method according to claim 38, wherein the positively charged backbone comprises polylysine having covalently attached to both ends one or more positively charged efficiency groups having an amino acid sequence selected from (gly)ₚ-RGRDDRRQRRR-(gly)_{q} (SEQ ID NO: 1), (gly)ₚ-YGRKKRRQRRR-(gly)_{q} (SEQ ID NO: 2) or (gly)ₚ-RKKRRQRRR-(gly)_{q} (SEQ ID NO: 3), wherein the subscripts p and q are each independently an integer of from 0 to 8.

48. The method according to claim 47, wherein the positively charged carrier has the amino acid sequence RKKRRQRRRG-(K)₁₅-GRKKRRQRRR (SEQ ID NO: 4) or RRRQRRKKRG-(K)₁₅-GRRRQRRKKR.

49. The method according to claim 37, wherein the total treatment dose of botulinum toxin is administered to the individual in an amount of 250 U to 500 U.

50. The method according to claim 37, wherein the duration of treatment effect comprises greater than 16 weeks to 26 weeks.

51. The method according to claim 37, wherein the risk of muscular weakness from the treatment is less than 5%.

52. The method according to claim 37, wherein the composition comprises a 150 kD botulinum toxin of serotype A, the positively charged carrier, a non-reducing disaccharide or a non-reducing trisaccharide selected from sucrose, trehalose and raffinose, the non-ionic surfactant selected from polysorbate 20, polysorbate 40, polysorbate 60, polysorbate 80, and a sorbitan ester, and a physiologically compatible buffer selected from citric acid, acetic acid, succinic acid, tartaric acid, maleic acid, and histidine for maintaining the pH between 4.5 and 6.8.

53. The method according to claim 37, wherein the composition comprises a 150 kD botulinum toxin of serotype A, the positively charged carrier, a non-reducing disaccharide or a non-reducing trisaccharide selected from trehalose, polysorbate 20, and histidine buffered to a pH between 4.5 and 6.8.

54. The method according to claim 37, wherein the total treatment dose of botulinum toxin is administered to the individual in an amount of 175 U to 350 U.


SEQUENCE LISTING (10 sequences)

SEQ ID NO: 1 - protein, 51 aa, Artificial Sequence
  synthetic construct; protein transduction domain
  misc (1)..(20): Gly at positions 1-20 can be present or absent
  misc (32)..(51): Gly at positions 32-51 can be present or absent
GGGGGGGGGGGGGGGGGGGGRGRDDRRQRRRGGGGGGGGGGGGGGGGGGGG

SEQ ID NO: 2 - protein, 51 aa, Artificial Sequence
  synthetic construct; protein transduction domain
  MISC (1)..(20): Gly can be present or absent
  MISC (32)..(51): Gly can be present or absent
GGGGGGGGGGGGGGGGGGGGYGRKKRRQRRRGGGGGGGGGGGGGGGGGGGG

SEQ ID NO: 3 - protein, 49 aa, Artificial Sequence
  synthetic construct; protein transduction domain
  misc (1)..(20): Gly can be present or absent
  misc (30)..(49): Gly can be present or absent
GGGGGGGGGGGGGGGGGGGGRKKRRQRRRGGGGGGGGGGGGGGGGGGGG

SEQ ID NO: 4 - protein, 35 aa, Artificial Sequence
  synthetic construct; protein transduction domain
RKKRRQRRRGKKKKKKKKKKKKKKKGRKKRRQRRR

SEQ ID NO: 5 - protein, 45 aa, Artificial Sequence
  synthetic construct; protein transduction domain
  MISC (1)..(20): Gly is present or absent
  MISC (26)..(45): Arg is present or absent
GGGGGGGGGGGGGGGGGGGGRRRRRRRRRRRRRRRRRRRRRRRRR

SEQ ID NO: 6 - protein, 19 aa, Artificial Sequence
  synthetic construct; Antennapedia PTD
SGRQIKIWFQNRRMKWKKC

SEQ ID NO: 7 - protein, 39 aa, Artificial Sequence
  synthetic construct; positively charged carrier
YGRKKRRQRRRGKKKKKKKKKKKKKKKGYGRKKRRQRRR

SEQ ID NO: 8 - protein, 39 aa, Artificial Sequence
  synthetic construct; protein transduction domain
RGRDDRRQRRRGKKKKKKKKKKKKKKKGRGRDDRRQRRR

SEQ ID NO: 9 - protein, 20 aa, Artificial Sequence
  synthetic construct; polylysine polypeptide
  misc (11)..(20): Lys is either present or absent
KKKKKKKKKKKKKKKKKKKK

SEQ ID NO: 10 - protein, 10 aa, Artificial Sequence
  synthetic construct;  protein transduction domain
GGGRRRRRRR